(19) United States
(12) Patent Application Publication
(10) Pub. No.: US 2006/0026704 A1
(43) Pub. Date: Feb. 2, 2006

(54) Vector for the production of transplastomic angiosperm plants

(76) Inventors: Guillermo Selman-Housein Sosa, Playa (CU); Eduardo Aguiar Cabeza, Sancti Spiritus (CU); Annery del Carmen Gonzales Quintero, Sancti Spiritus (CU); Osmany Ramos Gonzales, Lisa (CU)

Correspondence Address: Ronald J Baron;Hoffmann & Baron, 6900 Jericho Turnpike, Syosset, NY, 11791, US

(21) Appl. No.: 10/527,048

(22) PCT Filed: Sep. 15, 2003
(86) PCT No.: PCT/CU03/00009

§ 371 Date: Aug. 17, 2005

(30) Foreign Application Priority Data
Sep. 27, 2002 (CU) 2002-0208

(51) Int. Cl. A01H 1/00; A01H 5/00; C12N 5/04; C12N 15/82

(52) U.S. Cl. 800/278; 800/320; 800/320.1; 800/320.2; 800/320.3; 435/468; 435/419

(57) ABSTRACT

A DNA vector is provided for stable insertion and expression of heterologous genes in the plastid genomes of any Angiosperm plant cell. In this vector, foreign genes can be inserted in multiple cloning sites located at an artificial intergenic region obtained from the combination of atpB and rbcL operons that belong to plants of different Classes (dicotyledonous and monocotyledonous). The expression cassette is inserted into the plastid genome by homologous recombination between the atpB and rbcL border sequences of the vector with the corresponding homologous regions of plastid genome; in such a way that more than one gene of interest can be expressed under the transcriptional control of the rbcL promoter adjacent to the border region that encodes for the atpB gene.

### FIELD OF THE INVENTION

This invention is related to the field of Biotechnology, and more specifically to the Genetic Engineering of Plants. In particular, here are provided DNA constructs useful for the efficient introduction and expression of foreign genes in Angiosperm plant plastids. The usefulness of these genetic constructions to obtain with high efficiency transplastomic plants that express heterologous proteins, particularly vaccine antigens and proteins of pharmaceutical use is demonstrated.

### BACKGROUND OF THE INVENTION

The genetic engineering of plants is a technology that has shown to be very productive for basic research and commercial production of new biotechnological products (Hammond J. Curr. Top. Microbiol. Immunol 1999, 240:1-19; Simoens C. and Van Montagu M. Reproduction Update 1995, 1:523-542).

Until some years ago, the production of transgenic plants by direct or Agrobacterium-mediated transformation methods, was focused to the stable insertion of the chimeric genes into the nucleus of the plant cell (see: Hansen G. and Chilton M. D. Curr. Top. Microbiol. Immunol. 1999, 240:21-57; Newell C. A. Mol. Biotechnol 2000, 16:53-65). However, some limitation that present the nuclear transformation can be only overcome by the selective introduction of the new genes into the plant cell organelles (see: Bogorad L. Trends in Biotechnology 2000, 18:257-263; Heifetz P. B. Biochimie 2000, 82:655-666). In particular, the expression of heterologous genes in plastids presents the following advantages with regard to nuclear expression: 1) the expression level of the transgene is 10 to 50 times higher than in the nucleus due to high copy number of plastid genomes per cell; 2) the insertion of the new genes by homologous recombination is site-specific, avoiding positional effects, gene silencing and others undesirable phenomena that affect expression of genes introduced into the plant nucleus; 3) policistronic gene expression is possible, which is very desirable when trying to make engineering of metabolic pathways or altering multigenic characters; 4) the plastid genetic information is mainly maternally inherited, reducing the risk of possible damages to Nature due to the spreading of chimeric genes in the environment through pollen. Additionally, gene expression in plastids offers the possibility of accumulating heterologous proteins in a new microenvironment that could provide a greater stability and/or an easier purification process.

Different methods have been reported for the introduction of genes into plastids of algae and higher plants. In particular, it has been frequently used the bombardment with accelerated microparticles covered with DNA (Boynton J. E; Gilham E. H; Hosler J. P; and others. Science 1988, 240:1534-1538; Daniell H; Vivekananda J; Nielsen B; and others. Proc. Natl. Acad. Sci. USA 1990, 87:88-92; Svab Z; Hajdukiewicz P; Maliga P. Proc. Natl. Acad. Sci. USA 1990, 87:8526-8530), and in less proportion the transformation of plant protoplasts by PEG (O'Neill C; Horvath G. V; Horvath E; and others. Plant J 1993, 3:729-738; Golds T; Maliga P; Koop H-U. Biotechnology 1993, 11:95-97), and the microinjection (Knoblauch M; Hibberd J. M; Gray J. C; van Bel A. J. E. Nature Biotechnol 1999,17:906-909). The stable insertion of the introduced DNA sequences into plastid genome has been achieved often by using the efficient mechanisms of homologous recombination that naturally occur in these cellular organelles (Blowers A. D; Bogorad L; Shark K. B.; Sanford J. C. Plant Cell 1989, 1:123-132; Staub J. M. and Maliga P. Plant Cell 1992, 4:39-45; Kavanagh T. A; Thanh N. D; Lao N. T; and others. Genetics 1999, 152:1111-1122). It is important to emphasize that the larger the DNA regions with homology the higher the frequency of the recombination events, being these negligible when the homologous regions are smaller than 137 bp (Zoubenko O. V; Allison L. A; Svab Z; Maliga P. Nucleic Acid Res 1994, 22:3819-3824).

Many regions have been proposed as suitable recombination sites for inserting chimeric sequences in plant plastids: rbcL-accD, psbE-petA, trnV-16SrRNA operon, psbA-trnH, trnA-trnI, 23SrRNA region, etc (Svab Z. and Maliga P. Proc. Natl. Acad. Sci. USA 1993, 90:913-917; Zoubenko O. V; Allison L. A; Svab Z; Maliga P. Nucleic Acid Res 1994, 22:3819-3824; Daniell H; Datta R; Varma S; and others. Nature Biotechnol 1998, 16:345-348; U.S. Pat. No. 5,877,402; WO 9910513). However, in spite of some of these regions have been suggested as “universals” for the integration of foreign DNA sequences in plastid genomes of different plant species, their universality presents limitations due to one or many of the following reasons: 1) their sequences and/or structures are not highly conserved among the plastid genomes of different plant species; 2) they posses very short regions of high homology followed, or interrupted by, introns or others regions much less conserved (which decreases the homologous recombination frequency and therefore the obtaining of plants with transgenic plastids (transplastomics)); 3) they are located in some cases forming part of plastid operons, and it is known that an alteration inside of these structures can affect the wild transcription level for each operon's gene or the correct processing of policistronic RNA (Ohme M; Kamogashira T; Shinozaki K; Sugiura M. Nucleic Acid Res 1985, 13:1045-1056), with the undesirable consequences this causes to plastid metabolism. In fact, it is not fortuitous that using vectors containing the regions above mentioned as recombination sites, only there have been reported in peer-reviewed publications the production of transplastomic plants from tobacco (Svab Z. and Maliga P. Proc. Natl. Acad. Sci. USA 1993, 90:913-917), potato (Sidorov V. A; Kasten D; Pang S. Z; and others. Plant J 1999, 19:209-216) and tomato (Ruf S; Hermann M; Berger I. J; and others. Nature Biotechnol 2001, 19:870-875), all them Solanaceus species very close evolutionarily and at level of the DNA primary structure. In addition, vectors that use these recombination regions show a low frequency of production of homoplasmic transplastomic plants (plants with all their plastid genomes homogeneously transformed), probably because these vectors include the use of terminator regions and promoters of plastid origin larger than 174 bp (Iamtham S. and Day A. Nature Biotechnol 2000, 18:1172-1176), which causes that many transformation events fail due to recombination between these vector elements and the homologous regions of the plastid genome, giving rise to non viable plastids (Svab Z. and Maliga P. Proc. Natl. Acad. Sci. USA 1993, 90:913-917; Kanevski I; Maliga P; Rhoades D. F; Gutteridge S. Plant Physiol 1999, 119:133-141).

The region of the plastid genome more universally conserved by structure and nucleotide sequence between Angiosperm plants is that composed by the divergently transcribed rbcL and atpB operons. In fact, these genes contain DNA segments larger than 1500 bp with homologies superior to 85% between plastids of plants belonging to different classes (dicotyledonous or monocotyledonous) and more than 91% of nucleotide homology when analyzing plants of the same class (see Table 1). On the other hand, the intergenic region between these operons presents a very low nucleotide homology (less than 50%) among the plastids genomes of dicotyledonous and monocotyledonous plants.

TABLE 1
Nucleotide homology between the rbcL and atpB genes
of plants from different species and classes.

| | Maize | Tobacco | Rice | Spinach | |
|---|---|---|---|---|---|
| Maize | | 86.0% | 95.5% | 86.2% | atpB |
| Tobacco | 86.1% | | 86.6% | 91.8% | |
| Rice | 95.1% | 86.7% | | 87.0% | |
| Spinach | 86.2% | 91.1% | 85.8% | | |

rbcL

The large subunit of the ribulose-1,5-bisfosfate carboxilase-oxigenase (Rubisco) is codified by the plastid gene rbcL. It has been determined experimentally that the carboxyl-terminal region of rbcL subunit forms the alpha-helix number 8 of this protein, and that this region is involved in the interaction with the small subunit of this enzyme, codified by the nuclear gene rbcS (Knight S; Andersson I; Branden C-I. J. Mol. Biol. 1990, 215:113-160; Curmi P. M. G; Cascio D; Sweet R. M; and others. J. Biol. Chem. 1992, 267:16980-16989). Starting from these and others data (Kanevski I; Maliga P; Rhoades D. F; Gutteridge S. Plant Physiol. 1999, 119:133-141), and considering the small degree of conservation between C-terminal regions of plastid rbcL proteins belonging to different plant species, it was put forward the hypothesis that the alpha-helix 8 of rbcL defines the species-specificity of the interactions between both subunits of Rubisco enzyme.

It has been shown that possibly due to a loop-like structure that is located near to the transcription start of rbcL promoter (position +1 to +26 starting from the beginning of transcription) the produced messenger RNA become stable, that way compensating the decrease in transcription activity of this promoter during the darkness (Shiina T; Allison L; Maliga P. The Plant Cell 1998, 10:1713-1722). Likewise, various segments of sequences sited near or inside the structural region of this promoter resemble sequences of chloroplast DNA binding factors (CDF), and could play an important role in transcriptional regulation of the rbcL promoter.

Some features that characterize the transcription/translation of atpB-atpE operon (which encodes for β and ε subunits of the chloroplast ATP-synthase) have been reported (Shinozaki K; Deno H; Kato A; Sugiura M. Gene 1983, 24:147-155; Gatenby A. A; Rothstein S.; Nomura M. Proc. Natl. Acad. Sci. USA 1989, 86:4066-4070; Kapoor S; Wakasugi T; Deno H; Sugiura M. Curr. Genet. 1994, 26:263-268). These two genes are overlapped and in particular, the translational features of atpE call the attention because they are based on a frame-shift of the open reading frame, and on a conserved region inside the atpB coding sequence which contains a possible promoter for atpE (positions +1027 to +1064 of the nucleotide sequence that encodes for atpB protein of rice, tobacco, etc.). The functionality of this promoter “in vivo” has not been proved, but it is not discarded that it could play some role in transcriptional regulation of atpE expression.

The possible use atpB-rbcL intergenic region, as target for the insertion of foreign genes in plastids, was only described by Cannon M. C. and Cannon F. C in the European Application Patent No. 0251654. However, this patent proposed the use of a bacterial transposon as carrier molecule for introducing foreign genes into the intergenic region between these two divergently transcribed operons. It is not casual that Cannon M. C. and Cannon F. C. did not succeeded in obtaining transplastomic plants, because the insertion of a transposon in this intergenic region not only interrupts the reported interaction between atpB and rbcL promoters (Hanley-Bowdoin L. and Chua N-H. Mol. Gen. Genet. 1989, 215:217-224), but also (as it is for maize and rice, for example) it interrupts the transcription of the atpB gene starting from the NEP promoter (nuclear polymerase dependent promoter) that is located inside the 5′ untranslated region of the rbcL gene (Welhe A. and Borner T. Trends in Plant Science 1999, 4:169-170). It is evident that an accurate transcription and regulation of expression of the products codified by these operons are absolutely necessary for the correct functionality and viability of plastids. Additionally, the expression vector described by Cannon M. C. and Cannon F. C. does not contain transcription terminators, an indispensable element for proper gene expression in plastids (Stern D. B. and Kindle K. L. Mol. Cell Biol. 1993, 13:2277-2285; Chen H. C. and Stern D. B. Mol Cell Biol. 1991, 11:4380-4388).

In addition to transcription terminator regions, which can be of bacterial origin (Chen L. J. and Orozco E. M. Jr. Nucleic Acid Res. 1988, 16:8411-8431), it is necessary to put the genes introduced into plastids under the control of functional promoters in this organelle. Among the most used promoters for expressing genes in transgenic plastids are the strong promoters Prrn of 16S rRNA (Svab Z. y Maliga P. Proc. Natl. Acad. Sci. USA 1993, 90:913-917) and PsbA (Daniell H. Methods in Molecular Biology 1997, 62:463-489). The Prrn promoter is constitutive, while the transcription/translation of RNA produced by the PsbA promoter is induced by light and depends on its 5′ untranslated region. Deleting the 5′ untranslated region of the psbA gene makes its mRNA expression light independent, although this affects the translation (Staub J. M. y Maliga P. Plant J. 1994, 6:547-553; Eibi C; Zou Z; Beck A; and others. Plant J. 1999, 19:333-345). These promoters have −35 and −10 consensus sequences homologous to those described for bacterial promoters and their transcription is dependent of a polymerase codified by the plastid genome (PEP) (Hess W. and Borner T. Int. Rev. Cytol. 1999, 190:1-59).

Among others similarities between transcriptional/translational mechanisms of plastids and bacteria, it has also been proved that policistronic mRNA can be expressed and efficiently translated in tobacco plastids (Staub J. M. and Maliga P. Plant J. 1995, 7:845-848);—being functional in chloroplasts the ribosome binding sites (RBS) that comprise Shine-Dalgarno like sequences (Stem D. B; Higgs D. C; Yang J. Trends Plant Sci. 1997, 2:308-315).

To facilitate the selection of transplastomic plants, it has been proposed among others alternatives the use of positive selection markers as for example: kanamycin (Daniell H. Methods in Molecular Biology 1997, 62:463-489), or the most universally used spectinomycin (Svab Z; Hajdukiewicz P; Maliga P. Proc. Natl. Acad. Sci. USA 1990, 87:8526-8530; Svab Z. and Maliga P. Proc. Natl. Acad. Sci. USA 1993, 90:913-917); for this purpose the gene that confers resistance to the selection agent is cloned in the transformation vector under appropriate controlling signals for its correct expression in plastids.

To protect the Nature, there have been designed “clean” technologies that allow the removal of genes that encode for selection markers from plants that are released to the environment. Two of these technologies successfully applied to the production of transplastomic plants are the following: co-transformation with two vectors and the subsequent segregation and elimination of individuals that carry the selection marker (Fisher N; Stampacchia O; Redding K; Rochaix J. D. Mol. Gen. Genet. 1996, 251:373-380), and the insertion of marker genes between two directly repeated sequences in the expression cassette to allow the removal of these genes by homologous recombination during subsequent culture cycles in non selective medium (Fisher N; Stampacchia O; Redding K; Rochaix J. D. Mol. Gen. Genet. 1996, 251:373-380; Iamtham S. and Day A. Nature Biotechnol. 2000, 18:1172-1176).

### DETAILED DESCRIPTION OF THE INVENTION

The vector for stable transformation and expression of heterologous genes in plastids of Angiosperm plants that proposes the present invention comprises some own features that distinguish it: 1) it is not use a transposon to insert DNA fragments into plastid genomes; 2) the atpB and rbcL border regions belong to Angiosperm plants of different classes, forming an artificial intergenic region; 3) multiple cloning sites in the artificial intergenic region transcriptionally inactive, allow the insertion of one or several heterologous genes without affecting the correct transcription, mRNA processing and the expression of the atpB and rbcL genes; 4) the design of this vector allows the expression of introduced genes without the necessity of carrying promoter and terminator regions, whenever the sequences that encodes for protein are preceded by a ribosome binding site (this contribute to reduce undesirable recombination events and to save plastid metabolic resources, allowing a greater stability of the introduced heterologous DNA fragments and an increase in the production frequency of homoplasmic plants); 5) the structure and sequence of border regions proposed by the present invention, guarantee the vector universality. Additionally, the vector herein provided constitutes a “clean” tool for introducing genes into crop plants due to a design that allows the removal of selection marker, by homologous recombination between directly repeated sequences, once the homoplasmic transplastomic plants have been obtained.

The design and extension of the atpB-rbcL region, with its artificial intergenic region, constitute the base of the present invention object, since it allows us to insert genes and operons in an efficient and stable way into plastid genomes of Angiosperm plants of any species, without causing a functional alteration.

For designing the artificial intergenic region it was necessary to resolve the challenge that represents the introduction of a foreign gene in this region, without affecting the interaction between rbcL and atpB promoters, and their wild transcriptional levels.

On the other hand, it was also necessary to consider that the atpB and rbcL gene products constitute subunits of enzymes that carry out vital functions for the maintenance of plastids and plants. This situation imposes limitations to the length of fragments selected as borders for recombination, because it is advisable that certain domains of these proteins will be involved in protein-protein interactions and could determine the specificity of recognition between different subunits of these enzymes and their correct folding and functioning.

To use the atpB-rbcL region as a recombination site for foreign genes insertion in plastids of different plant species, it is important to consider that hybrid proteins will be produced, and therefore what it was mentioned above deserves an especial attention. In this way, to avoid interferences with the correct folding and functioning of hybrid Rubisco, it was decided to maintain invariable during the recombination event the rbcL gene region that encodes for the alpha helix 8. Thus, during the design of the vector aim of this invention it was decided that the DNA fragment for plastid transformation by homologous recombination, containing part of the translatable rbcL gene sequence (further called rbcL-border), should extend only till N-terminal region of alpha helix 8, and more specifically till a tryptophan (Trp-411) highly conserved in this position among rbcL proteins from Angiosperm plants.

Concerning the region that encodes for the atpB gene, it is not detected any domain with a marked variability when comparing protein sequences from plastids of different plant species. However, the transcription/translation features of atpB-atpE operon were taken into consideration to select the DNA fragment, containing part of the translatable atpB gene sequence, (further called atpB-border) for the integration of foreign genes into plastids by homologous recombination.

To design the artificial intergenic region that allows the insertion of new genes into plastids without affecting the atpB and rbcL transcription rates, we took advantage of the low homology at nucleotide level that exhibit the region between these operons when comparing plastids sequences belonging to Angiosperm plants of different species.

In the genetic construction aim of this invention, the rbcL-border should comprise a DNA fragment that bears part of the gene and the whole 5′ regulatory region. This fragment includes: 1) the rbcL gene PEP promoter (dependent on plastid encoded polymerase) with its associated CDF sequences; 2) the 5′ untranslated region of the rbcL gene and 3) the sequence that encodes for the rbcL protein till the region that corresponds to the N-terminal of alpha helix 8. Thus, the rbcL-border in the vector aim of this invention comprises a DNA fragment which extends from the position −291 of nucleotide sequence that encodes for this protein in tobacco, to position +1233 (Trp-411 in the aminoacid sequence) starting from the translation start codon of this gene.

It is necessary to point out when attempting to clone the rbcL-border, we discovered that expression of the rbcL gene starting from its own promoter is toxic to E. coli. Therefore, it was necessary to replace a little conserved fragment from the 5′ untranslated region of this gene for an “ideal” lac operator (Simons A; Tils D; von Wilcken-Bergmann B; Muller-Hill B. Proc. Natl. Acad. Sci. USA 1984, 81:1624-1628) and also, to clone this chimeric border into a low copy number plasmid vector (pBR322) that will be propagated in a lacl^{q} E. coli strain, as for example XL-I blue, JM 101, etc.

In the genetic construction aim of this invention it was decided to take as atpB-border a DNA fragment that includes: 1) the adjacent rbcL promoter and the stem-loop forming sequences near to the transcription start point that stabilize the mRNA produced by this promoter; 2) the promoters of the atpB gene including its NEP promoter; 3) the 5′ untranslated region of the atpB gene and 4) the sequence that encodes for atpB till 300 bp before the stop codon of the gene. In this way, when designing the vector aim of this invention the atpB-border extends from position −654 of nucleotide sequence that encodes for this protein in rice, to position +¹²¹I starting from the translation start codon of this gene. In the atpB-border, the rbcL promoter adjacent to atpB gene allows the stable transcription and expression of genes of interest in opposite direction to the transcription of the atpB-atpE operon. At user's convenience, under this rbcL promoter could be possible to clone a genetic cassette containing one or more foreign genes controlled by a promoter functional in plastids, so the expression of these genes will be enhanced by the transcription from two promoters in tandem.

The borders regions previously described are introduced into a plasmid vector, for their maintenance and multiplication in E. coli, separated by a DNA segment that contains multiple cloning sites (MCS 1, see FIG. 1-A), useful for insertion of heterologous genes that further will be introduced into plastid genomes. According to this structure, in which the atpB and rbcL genes will be divergently transcribed and due to rbcL promoter region is duplicated, two directly repeated DNA fragments are obtained bordering the region that contains the cloning sites. However, owing to the low homology at nucleotide level in this intergenic region between plastid genomes from different Angiosperm plant species, it is not foreseen any recombination event in our construction because only a small fragment of 90 bp has a high degree of homology.

The construction previously described is the base of the vector aim of this invention, where a gene encoding for a selectable marker that allows the positive selection of transformed cells will be introduced into the MCS1 (in direction to the rbcL-border, starting from the rbcL promoter of the atpB-border), bordered by directly repeated DNA sequences that will enable the removing of the marker gene by homologous recombination, once homoplasmic transplastomic plants have been obtained. The transcription termination of the marker gene, or other gene introduced into the MCS1, will be carried out by a bacterial terminator located adjacent to the 5′ end of the rbcL-border (preferably the rrnBT1T2 bi-directional terminator of rrnB operon of E. coli (Brosius J; Dull T. J; Sleeter D. D; Noller H. F. J. Mol. Biol. 1981, 148:107-127), thus avoiding that transcription of inserted genes interfere the normal functioning of the rbcL-border promoter.

In this way, the general structure of the vector aim of this invention is shown in FIG. 1-B, where the gene o genes of interest are inserted under the rbcL promoter of the atpB-border forming a transcriptional unit mono or polycistronic (FIG. 1-C), or under other plastid functional promoters allowing the obtainment of, for example, a polycistronic unit under two promoters in tandem (FIG. 1-D), or two independent transcriptional units under different combinations of promoter regions (FIG. 1-E and FIG. 1-F).

For facilitating the selection of transplastomic plants, it was decided to use the hygromycin as selection marker; although this antibiotic is extremely toxic to dicotyledonous plants as tobacco, and therefore it should be used carefully, it results more effective as selection agent for the obtainment of monocotyledonous plants with genetically modified plastids. In this way, the hgh gene that encodes for resistance to hygromycin B in Klebsiella (Gritz L. and Davies J. Gene 1983, 25:179-188), containing an appropriate Shine-Dalgarno region, is cloned into the MCS 1 under rbcL promoter of the atpB-border (FIG. 1-B).

With the purpose of offering an ecological tool, in the vector aim of this invention the marker gene was introduced bordered by two directly repeated DNA sequences that allow its removing by homologous recombination (FIG. 1-B), once the selective agent is eliminated from the culture media when the homoplasmic transplastomic plants have been already obtained. The repeated sequences used in this construction came from a DNA region that encodes for the s10 protein of phage T7 (Dunn J. J. and Studier F. W. J. Mol. Biol. 1983, 166:477-535). These vectors have the peculiarity of removing only the marker gene, so that the rest of operon regulatory sequences are maintained invariable (FIGS. 1-C, 1-D, 1-E and 1-F); therefore, any gene transcriptionally fused to marker gene will unchangeable continue its expression, in this way economizing the vector resources. This architecture is another of the qualities that distinguish the vector aim of this invention from others described systems as vehicles for genetic transformation of plastids.

It is obvious that others genes which confer resistance to antibiotics as to spectinomycin (Chinault A. C; Blaskeley V. A; Roessler E; Willis D. G; and others. Plasmid 1986, 15:119-131), or to sulphonamides (Guerineau F. and Mullineaux P. Nucleic Acid Res 1989, 17:4370), could be also used to allow the selection of transplastomic plants. Likewise, genes that confer resistance to herbicides like glyfosate (Shah D. M; Horsch R. B; Klee H. J; Kishore G. M; and others. Science 1986, 233:478-481; Comai L; Facciotti D; Hiatt W. R; Thompson G; and others. Nature 317:741-744), gluphosinate (DeBlock M; Botterman J; Vandewiele M; Dockx J; and others. EMBO Journal 1987, 6:2513-2518), asulam (Guerineau F; Brooks L; Meadows J; Lucy A; and others. Plant Mol Biol 1990, 15:127-136), bromoxinil (Stalker D. M. and McBride K. E. J. Bacteriol 1987, 169:955-960), sulphonilureas and imidazolinones (LaRossa R. A and Smulky D. R. J. Bacteriol 1984, 160:391-394), etc, can be cloned in the artificial intergenic region and used as selection markers with the advantage that herbicide tolerance is an agronomical desirable character.

For somebody skilled in the state of the art and genetic engineering techniques, it is obvious that what is described here allows the use of any combination of atpB and rbcL regions, with all or parts of the described features, as border regions for integration of genes into plastid genomes whenever these borders come from plastids of Angiosperm plants of different classes (monocotyledonous or dicotyledonous). Likewise, multiple genes, selection markers and combinations of cistrons and operons can be cloned into the MCS1 of the artificial intergenic region of the vector aim of this invention (FIG. 1-A), in order to stably insert and express them in plastids of any Angiosperm plant.

Deposit of Microorganisms.

The plasmid vectors pVTPA-HB-aadA, pVTPA-f-GUS and pVIEP were deposited under the Budapest Treaty Rules in the Belgian Coordinated collection of Microorganisms BCCM, LMBP-COLLECTION with the numbers LMBP 4635, LMBP 4636 and LMBP 4637 respectively deposited on Sep. 24, 2002.

### DESCRIPTION OF THE DRAWINGS

FIG. 1. A, basic structure for the construction of the vector aim of the present invention showing the region that contains the multicloning sites (MCS1) limited by: [symbol] atpB border and [symbol] rbcL border belonging to Angiosperm plants of different classes (monocotyledonous or dicotyledonous).

B, general structure of the vector aim of the present invention (rbcLpr-promoter of the rbcL gene, atpBpr-promoter of the atpB gene, Term-transcription terminator of non-plastid origin, marker-selection marker gene, R1, R2-direct repeated sequences that allow the removing of marker gene by homologous recombination).

C, D, E, F, variants of the vector aim of the present invention showing the possibilities of expressing one or more genes of interest (gene, gene1, gene2) as monocistronic units (E, F), bicistronics (C, E, F) or tricistronics (D), under the control of one (C, E, F) or more (D, F) plastid functional promoters (Pr, Pr1, Pr2).

FIG. 2. Map of the vector for transformation of plastids of Angiosperm plants, pVTPA-f.

FIG. 3. Map of the vector for transformation of plastids of Angiosperm plants, pVTPA.

FIG. 4. Map of the intermediary vector for the expression of genes in plastids, pVIEP (S/D-Shine-Dalgarno sequence).

FIG. 5. Southern blot of 10 μg of chloroplast DNA purified by sucrose gradient, digested with PstI and using as probe the 5′ fragment of the rbcL gene labeled with ³²P. Lane 1: non-transformed plant; lanes 2-6: transformed plants (it can be appreciated that all plants are homoplasmic); lanes 7 and 8: transformed plants grown in absence of selection agent during the last cycle of in vitro tissue culture (it can be noted in lane 7 that the marker gene has not been removed totally).

FIG. 6. Western blot for demonstrating the use of the transformation vectors aim of this invention for expressing the Hepatitis B surface antigen in chloroplasts: 10 μg of total leaves proteins were applied in each lane. Lane 1: non-transformed plant; lanes 2-5: transplastomic plants expressing the HbsAg (it can be observed the signal revealed by the antibody against the Hepatitis B virus at the expected molecular weight of 25 kDa).

FIG. 7. Western blot for demonstrating the use of the transformation vectors aim of this invention for expressing in plastids the heavy and light chains that conform a monoclonal antibody: 10 μg of total leaves proteins were applied in each lane. Lane 1: non-transformed plant; lanes 2-5: transplastomic plants expressing the recombinant antibody CB-Hep.1 (it can be observed the signals corresponding to heavy (50 kDa) and light (25 kDa) chains of the immunoglobulin).

### REALIZATION EXAMPLES

### Example No. 1

### Construction of Vectors for Stable Transformation and Expression of Heterologous Genes in Plastids of Angiosperm Plants

a) Obtainment of rbcL-Border.

The rbcL-border fragment was obtained by using the oligonucleotides described in SEQ ID NO: 1 and SEQ ID NO: 2 for the amplification by polymerase chain reaction (PCR) of a 1524 bp DNA fragment corresponding to the gene that encodes for rbcL protein of tobacco. For the PCR was used chloroplast DNA purified from leaves of N. tabacum var. SR1. The amplified DNA fragment was cloned in pBScript SK vector (Stratagene, USA), previously digested with SpeI(Klenow)/EcoRV restriction enzymes, to produce the pBSrbcL clones. Some of the positive clones were totally sequenced and it was demonstrated that in spite of using a high fidelity DNA polymerase when performing PCR (Pfu DNA Polymerase, Promega, USA) all clones presented mutations. This fact and others evidences non-related to the present invention, indicated that rbcL protein of tobacco was toxic to E. coli when expressing from its own promoter. For this reason, it was decided to replace a low conserved DNA fragment of the 5′ untranslated region of rbcL gene by a DNA fragment that contains an “ideal” lac operator allowing to clone this border fragment in lacI⁺ E. coli strains. To perform this mutagenesis the pBSrbcL13 clone (one which contains a mutation in the SacII/BamHI fragment of cloned rbcL gene) was digested with NcoI and EcoRI restriction enzymes, at positions −162 and −29 respectively, and this fragment was replaced by a synthetic DNA fragment carrying an “ideal” lac operator inserted in the −124 to −97 region of the rbcL gene (SEQ ID NO: 3).

Once the lac operator was inserted in pBSrbcL13 clone (called now pBSrbcL13lacOp), the rbcL fragment was excised by XbaI(Klenow)/SalI restriction enzymes and it was cloned into a low copy number vector pBR322 (New England Biolabs, USA) previously digested with SalI and EcoRV restriction enzymes (XbaI site was restored), to obtain the plasmid pBR322rbcL13lacOp, where the rbcL gene transcription occurs in SalI/XbaI direction. Then, replacing the SacII/BamHI fragment of pBR322rbcL13lacOp by a mutationless SacII/BamHI fragment obtained from another sequenced pBSrbcL clone, we eliminated the mutation present in the rbcL13 clone. This way it was obtained the plasmid pBR322rbcLOK, containing the rbcL-border (SEQ ID NO:4).

b) Obtainment of atpB-Border.

The atpB-border fragment was obtained by using the oligonucleotides described in SEQ ID NO: 5 and SEQ ID NO: 6 for the amplification by PCR of a 1754 bp DNA fragment corresponding to the gene that encodes for plastid atpB protein of rice. For the PCR was used DNA purified from chloroplasts of Oriza sativa var. IAC-18. The amplified DNA fragment was cloned into pBScript KS vector (Stratagene, USA) previously digested with HincII restriction enzyme (the HincII(SalI) restriction site is restored at 5′ region of the cloned fragment), to produce the pBSatpB clones. Some of the positive clones were sequenced and it was chosen a totally correct clone.

The correct pBSatpB clone was digested with HindIII and SalI restriction enzymes at 5′ end of the fragment (the original sequence is restored and the SalI site is lost after cloning) for cloning a synthetic DNA fragment which carries additional restriction sites and part of the 5′ untranslated region of the rice atpB gene that contains the NEP promoter (SEQ ID NO: 7), obtaining the plasmid pBSatpBcomplete. In this vector the transcription of the atpB gene occurs in direction from HindIII to XhoI. The nucleotide sequence of the constructed atpB-border is shown in SEQ ID NO: 8.

c) Obtainment of Selection Cassette with Repeated Borders.

The 1 kb DNA fragment comprising the gene that encodes for hygromycin resistance (hgh) was obtained starting from the plasmid pBSBar, by PCR amplification using the oligonucleotides described in SEQ ID NO: 9 and SEQ ID NO: 10, which introduce a Shine-Dalgarno sequence 7 bp before the gene translation start, as well as restriction sites for facilitating the manipulation of this fragment. The amplified DNA fragment was digested with KpnI restriction enzyme and cloned into KpnI/SmaI-digested pUC19 vector (the SmaI site is conserved after cloning), giving rise to the plasmid pUC19Hyg. Subsequently, the obtained plasmid was digested with HindIII and SmaI restriction enzymes, and it was introduced a synthetic DNA fragment comprising restriction sites that will be useful for further genetic manipulations (SEQ ID NO: 11). The obtained plasmid vector was called pUC19-linker-Hyg.

The DNA fragment that will be repeated for eliminating the marker gene by homologous recombination once obtained the homoplasmic transplastomic plants, was obtained from a DNA fragment that encodes for the s10 protein of phage T7, present in the pET3xb expression vector (Novagen, USA). The pET3xb vector was initially digested with BamHI and NdeI, and the obtained 780 bp DNA fragment was re-digested with TaqI(Klenow) and KpnI, to produce a DNA fragment of approximately 310 bp that was cloned into SmaI/KpnI-digested pUC 19 vector, for obtaining the plasmid pUC-Spacer.

The s10 gene fragment of 310 bp from pUC19-Spacer was cloned downstream of the hgh gene as an XbaI(Klenow)/KpnI fragment in the plasmid pUC19-linker-Hyg previously digested with EcoRI(Klenow)/KpnI, giving rise to pUC19-linker-Hyg-Spacer. The second 310 bp repeated fragment was cloned upstream of the hgh gene, as an EcoRI(Klenow)/BamHI fragment, in the plasmid pUC19-linker-Hyg-Spacer previously digested with SmaI and BglII restriction enzymes, to produce the plasmid pUC-linker-Spacer-Hyg-Spacer.

The bi-directional terminator rrnBT1T2 from E. coli was obtained from the pTrcHisB expression vector (Invitrogen, USA), by digestion with BspHI, treatment with Mung Bean nuclease and HindIII digestion, for cloning the 470 bp terminator fragment into pBScript SK vector previously digested with HindIII and SmaI restriction enzymes, to obtain the plasmid pBS-rrnBT1T2.

Finally, the DNA fragment comprising the rrnBT1T2 terminator was cloned, as a HindIII(Klenow)/XbaI fragment, downstream of the Spacer fragment that is continuous to the hgh gene in the plasmid pUC-linker-Spacer-Hyg-Spacer previously digested with BamHI(Klenow)/XbaI, to produce the plasmid pUC-linker-Spacer-Hyg-Spacer-rrnBT1T2. The cassette comprising from linker to rrnBT1T2 was excised form the previous construction as a HindIII/XbaI fragment, and cloned in pBScript SK vector HindIII/XbaI-digested to obtain a plasmid with the selection cassette with repeated borders, pBS-linker-Spacer-Hyg-Spacer-rrnBT1T2 (SEQ ID NO: 12).

d) Construction of the Vector for Stable Transformation and Expression of Heterologous Genes In Plastids of Angiosperm Plants, pVTPA-f.

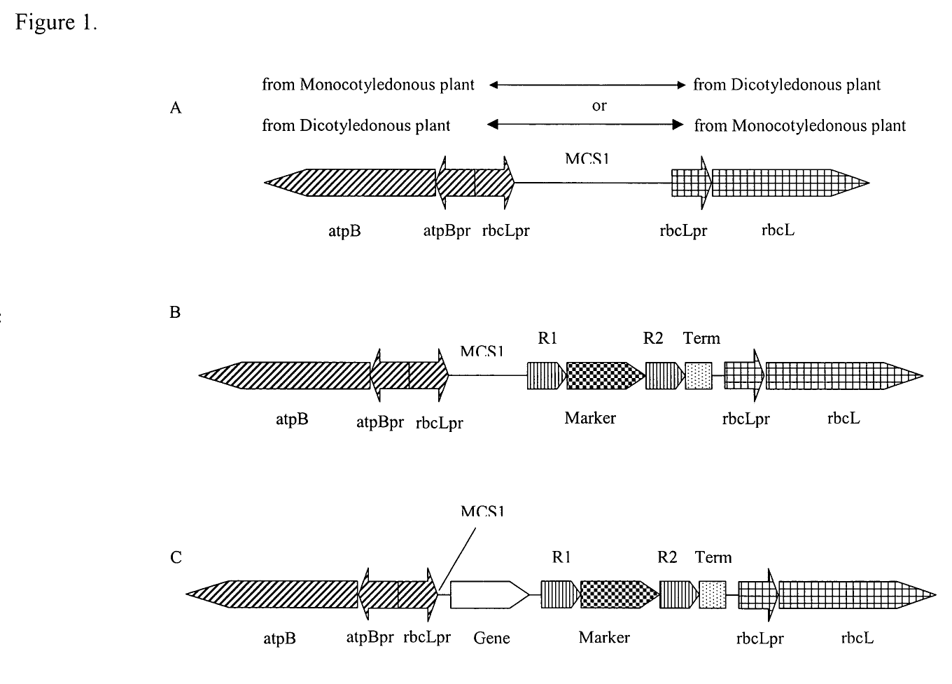

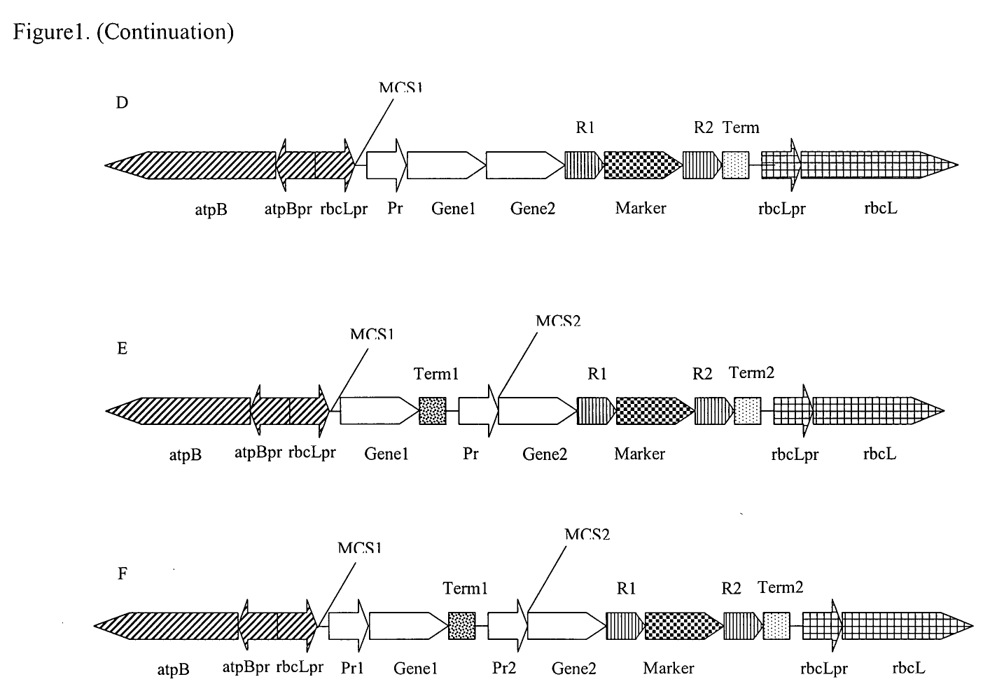

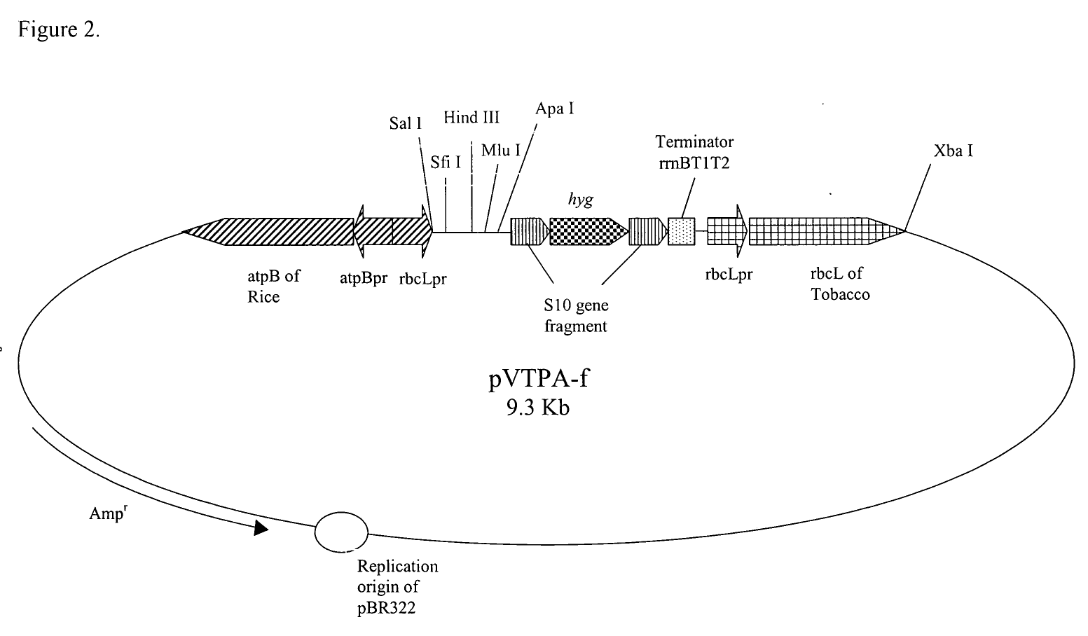

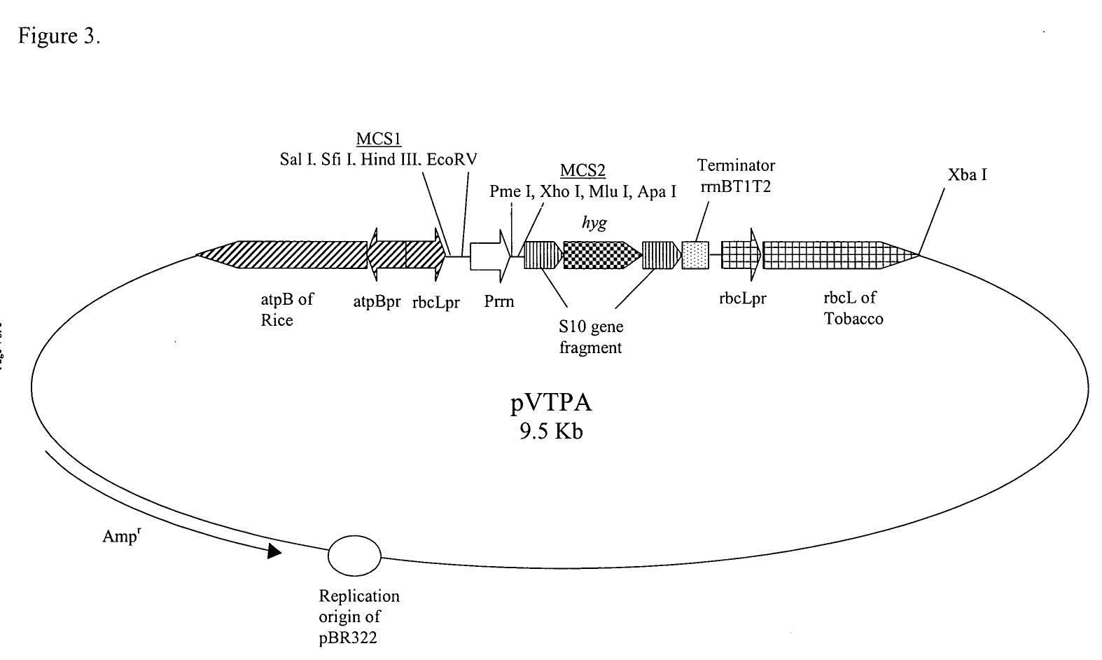

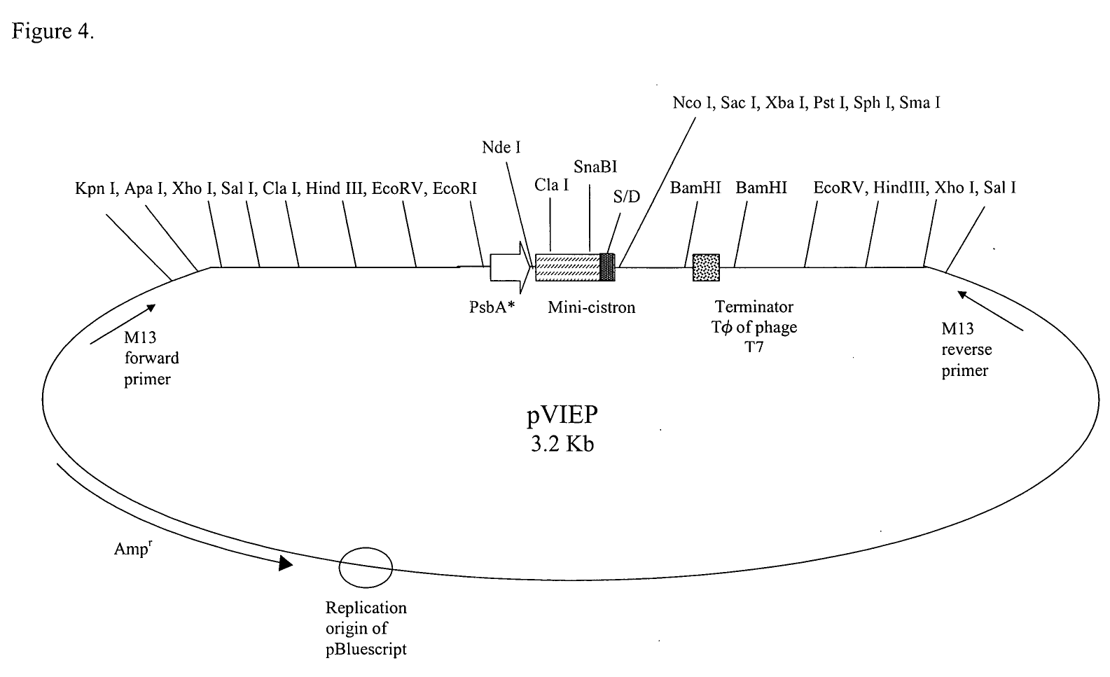

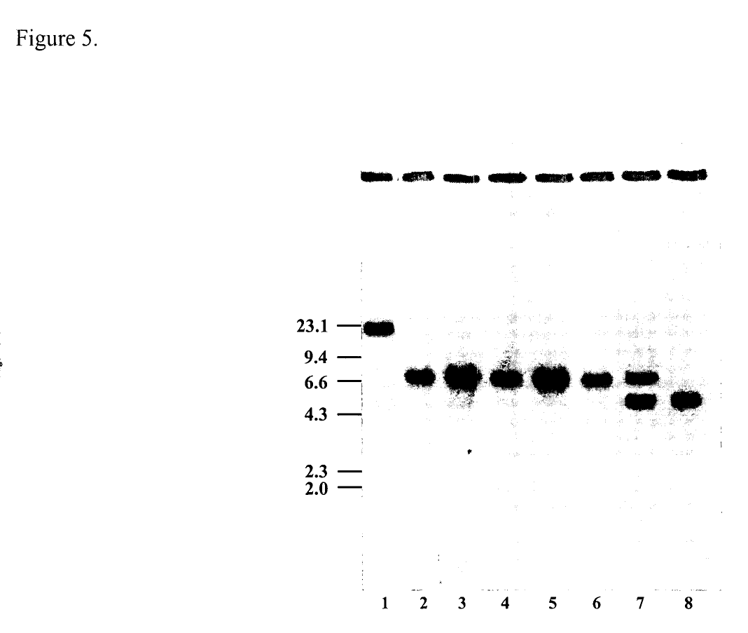

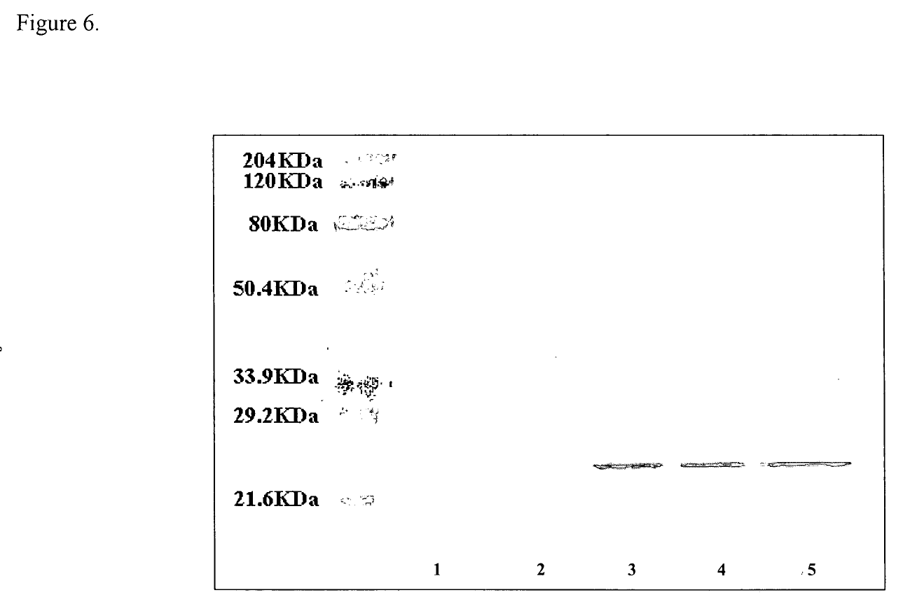

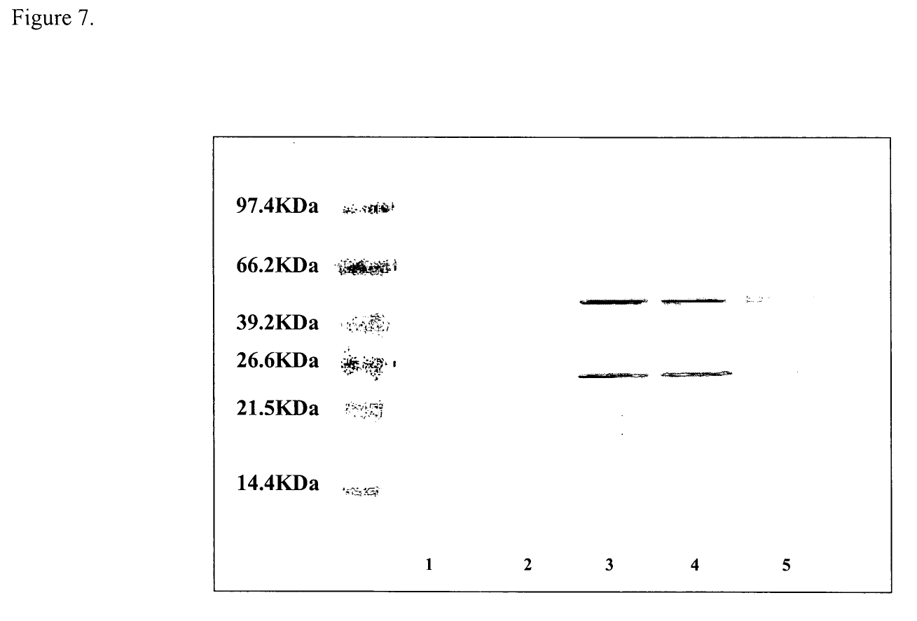

The vector for stable transformation and expression of heterologous proteins in plastids of Angiosperm plants that this invention proposes was assembled as follows: the selection cassette comprising the linker-Spacer-Hyg-Spacer-rrnBT1T2 was cloned as a NotI(Klenow)/SalI fragment in the vector pBR322 previously digested with EcoRI(Klenow)/SalI for producing the plasmid pBR322-sp-Hyg-sp-T. Subsequently, the rbcL-border from plasmid pBR332rbcLOK was cloned as a HindIII(Klenow)/XbaI fragment in the plasmid pBR322-sp-Hyg-sp-T digested with BamHI(Klenow)/XbaI, to obtain the construction pBR-sp-Hyg-sp-T-rbcL. Finally, the atpB-border from plasmid pBSatpBcomplete was cloned as a HindIII/XhoI fragment in the pBR-sp-Hyg-sp-T-rbcL digested with HindIII and SalI. So this way is obtained a vector with the structure described in FIG. 1-B, called pVTPA-f (see the map in FIG. 2). The nucleotide sequence of a DNA fragment from the pVTPA-f, between positions +651 (SalI site) and +1 (EcoRI site) of the pBR322 carrier vector, is shown in sequence SEQ ID NO: 13.

The features of pVTPA-f vector allow the stable transformation of Angiosperm plant plastids and the obtainment of transplastomic plants that express recombinant proteins encode by genes that were cloned in this vector, whenever these genes contain ribosome binding sites at a proper distance from their translation initiation codons.

e) Vector pVTPA: Variant of the Vector pVTPA-f that Allows the Insertion and Expression in Plastids of Several Genes as Independent Transcriptional Units.

The variant of the vector pVTPA-f that allows the insertion and expression in plastids of several genes as independent transcriptional units was obtained by inserting into cloning sites of the pVTPA-f vector DNA sequences able of promoting the transcription in plastids, as well as the termination of transcription (see FIG. 1-E). For this purpose as an example, a synthetic DNA fragment comprising the promoter of the 16S subunit of plastid ribosomal RNA (Prrn) (SEQ ID NO: 14), and containing cloning sites for facilitating genetic manipulations, was inserted between the EcoRI and SmaI restriction sites of the vector pBluescript SK, for obtaining the plasmid pBSPrnn. Subsequently, the DNA fragment comprising Prrn promoter was cloned as a HindIII/MluI fragment in plasmid pVTPA-f, giving rise to construction pVTPA (see FIG. 3 and sequence SEQ ID NO: 15).

### Example No. 2

### Expression of a Heterologous Monocistronic Unit in Tobacco Transplastomic Plants (pVTPA-f-GUS)

For expressing an heterologous gene in transplastomic plants by using the vector pVTPA-f, it is necessary that this gene carries a ribosome binding site (RBS) approximately 5 to 15 bp upstream of its translation initiation codon. For this reason, and with the aim to facilitate the cloning of foreign genes in our vector, it was designed an intermediary vector (pVIEP) that allows to insert herein the genes of interest and later excise them by restriction digestion before cloning in the vectors pVTPA-f or pVTPA. The cloning of genes in the pVIEP vector introduces a RBS to them, and at the same time allows the addition of a transcriptional terminator or an additional promoter for the correct expression in the vector aim of this invention.

For constructing the vector pVIEP, a DNA fragment comprising the promoter of tobacco psbA gene with part of its 5′ untranslated region deleted (between positions −68 and −25 starting from the initiation of translation) for avoiding the translation control of this gene by light (PpsbA*, SEQ ID NO: 16), was cloned as an EcoRI/SmaI fragment in the vector pBluescript SK, to obtain the plasmid pBSPpsbA*. Subsequently, a synthetic DNA fragment containing a mini-cistron (for stabilizing the mRNA and help its translation), a ribosome binding site (RBS) and multiple cloning sites (SEQ ID NO: 17) were cloned downstream of the PpsbA promoter as a NdeI/SacI fragment (SacI site is lost after cloning) in the plasmid pBSPpsbA*, giving rise to plasmid pBSPpsbA*-linker.

Finally, the pVIEP vector was obtained by inserting the phage T7 (Tθ) transcription terminator, obtained from plasmid pET3c (Novagen, USA) by BamHI/EcoRV digestion, in the plasmid pBSPpsbA*-linker BamHI/StuI-digested. The sequence SEQ ID NO: 18 corresponds to the primary structure of the pVIEP vector (see FIG. 4) between the KpnI and the second SalI sites.

A version of the intermediary vector pVIEP without the PpsbA* promoter and the mini-cistron was obtained by digesting pVIEP with EcoRI(Mung bean nuclease)/SnaBI, and re-ligating, to produce the construction pVIEP-2.

For expressing an heterologous monocistronic unit in transplastomic plants, the uidA gene (gus) was obtained from plasmid pDMC200 (CAMBIA) by digesting with NcoI/SmaI, and ligated into pVIEP-2 vector NcoI/SmaI-digested to obtain the plasmid pVIEP-GUS. A DNA fragment comprising the uidA gene fused to the RBS obtained from the plasmid pVIEP-GUS, was cloned downstream of rice rbcL promoter as a HindIII/SmaI fragment in the plastid transformation vector pVTPA-f MluI(Klenow)/HindIII-digested, to obtain the construction pVTPA-f-GUS (SEQ ID NO: 19). Transplastomic plants transformed with the pVTPA-f-GUS vector will express the genes hgh and uidA as a bi-cistronic unit under the transcriptional control of the rbcL promoter, while the selection is maintained during transformation stages; however, the marker gene is going to be removed by homologous recombination once the transplastomic plants are cultivated in non selection conditions, and the expression of the uidA gene will remain further from a monocistronic expression unit.

The functioning of this construction was demonstrated by obtaining tobacco transplastomic plants according to the following method:

Leaves of tobacco (var. SR1) from 4-6 weeks old plants grown in vitro were placed abaxial side up on shoot induction medium (SI: MS salts and vitamins (Murashige T., Skoog F. Physiol. Plant. 1962, 15:493-497), BAP 1 mg/L, NAA 0.1 mg/L, sucrose 25 g/L, phytagel 4 g/L, pH 5.7) supplemented with 0.4 M of manitol for an osmotic pre-treatment of 2-4 hours. Gold particles of 0.6 μm (BioRad) were coated with DNA for bombardment as published (Russell D. R., Wallace K. M., Bathe J. H., and others. Plant Cell Rep. 1993, 12:165-169). Transformation was performed by using the PDS-1000/He biolistic gun (Biorad) with a pressure of 1100 psi, a shoot distance of 6 cm and one shoot per leaf sample. Bombarded leaves were maintained on iso-osmotic medium for 14-16 hours and later were placed on SI medium for 2 days. Subsequently, leaves were cut into sections (3 mm×3 mm) and transferred with abaxial side down to SI medium containing 5 mg/L of Hygromycin B (Duchefa, Netherlands). Resistant green shoots and calli appear in 5-8 weeks of culture; green calli and shoots were transferred onto the same selective medium for a second and a third selection cycle using increasing concentrations of Hygromycin B (10 mg/L and 15 mg/L). Finally, resistant shoots were rooted on MS medium (MS salts and vitamins, sucrose 30 g/L, phytagel 4 g/L, pH 5.8) supplemented with 15 mg/L of Hygromycin B to obtain plants. All of the culture stages were carried out under a regime of 16 hours of light and 8 hours of darkness.

The GUS activity of some clones resistant to Hygromycin B was analyzed by histochemical assay using X-gluc according to Jefferson (Jefferson R. A. Plant Mol. Biol. Rep. 1987, 5:387-405). The results of 3 transformation experiments and their controls are shown in Table 2.

TABLE 2
Obtainment of transplastomic plants with the construction pVTPA-f-GUS.

| | No. of | No. of clones | |
|---|---|---|---|
| | bombarded | resistant to | GUS⁺ |
| Experiment. | leaves. | Hygromycin. | clones. |
| I | 10 | 11 | 11/11 |
| II | 10 | 14 | 14/14 |
| III | 10 | 9 | 9/9 |
| Total | 30 | 34 | 34/34 |
| Negative controls | 30 | 0 | — |
| (bombarded | | | |
| without plasmid) | | | |

Additionally, for some clones selected at random it was demonstrated the integration of the chimeric DNA fragment into chloroplast genomes by Southern blot (Sambrook J., Fritsch E. F., Maniatis T. 1989. Molecular cloning: a laboratory manual. 2^{nd} edition. Cold Spring Habor Laboratory Press) of chloroplast DNA purified by sucrose gradient, digested with PstI restriction enzyme and using as probe a labeled fragment of the rbcL gene (FIG. 5). All of the analyzed positive clones yielded the expected band of approximately 6.8 kb and the negative control (non transformed plant) yielded the expected band of 21.5 kb. When some of the transplastomic clones were propagated in non selective culture medium, it was observed in the Southern blot that these clones yielded a hybridizing band of 5.5 kb, which confirms the elimination of the marker gene (hgh) by homologous recombination between the direct repeated sequences that border the gene.

Some rooted transplastomic clones were transferred to greenhouse for obtaining seeds by self-pollination. The collected seeds were germinated in MS medium and MS supplemented with 15 mg/L of Hygromycin B, demonstrating the elimination of marker gene in these clones, whereas the GUS activity was maintained invariable, confirming this way the homoplasmic state of transplastomic plants.

It was also noted in our experiments that unlike the results obtained when using Spectinomycin as selection agent for plastid transformation experiments, we didn't obtain spontaneous mutants when using Hygromycin B as selection agent. However, it was necessary to establish the appropriate concentrations of selection agent for recovering transplastomic plants.

These experimental data confirm the functionality of the vector aim of this invention, and its usefulness for obtaining transplastomic plants that express genes of interest from any source.

### Example No. 3

### Expression of a Heterologous Monocistronic Unit in Tobacco Transplastomic Plants Under the Control of Two Plastid Functional Promoters (pVTPA-aadA)

By using the pVTPA vector it is possible to express in plastids a gene under the control of Prrn and rice rbcL promoters; for this purpose, the gene that encodes for the enzyme aminoglycoside 3′-adenylyltransferase (aadA, which confers resistance to the antibiotics Streptomycin and Spectinomycin) was amplified by PCR from vector pDE1001 (Department of Genetics, Gent University, Belgium) using the oligonucleotides described in sequences SEQ ID NO: 20 y SEQ ID NO: 21. The amplified DNA fragment was cloned into the vector pVTPA XhoI(Klenow)-digested under the transcriptional control of the two promoters mentioned above, giving rise to the construction pVTPA-aadA (SEQ ID NO: 22). The tobacco transplastomic plants were obtained according to the methodology described in the Example No. 2, with the peculiarity that transplastomic clones were selected by using Spectinomycin (500 mg/L) in the first selection cycle and Streptomycin (500 mg/L) during the second selection cycle. For comparing the frequency of obtaining transplastomic plants by using vectors with different features, we carried out some transformation experiments with the vector pVSR326MOD (ICGIB, New Delhi, India). This vector has inserted between the tobacco rbcL and accD genes two expression cassettes containing: the gus gene under regulation of the psbA gene promoter and terminator; and the aadA gene under the control of the Prrn promoter and the terminator of the rbcL gene. The results of these transformation experiments after two regeneration/selection cycles and three cycles of in vitro culture without selection are shown in Table 3.

TABLE 3
Comparison of transformation frequency between vectors
pVTPA-aadA and pVSr326MOD.

| | | | No. of | |
|---|---|---|---|---|
| | | No. of | clones | No. of clones |
| | | bom- | resistant to | per |
| | Genetic | barded | Specti- | bombarded |
| Experiment. | Construction. | leaves. | nomycin. | leaves. |
| I | pVTPA-aadA | 10 | 19 | 1.9 |
| | pVSR326MOD | 10 | 13 | 1.3 |
| II | pVTPA-aadA | 10 | 11 | 1.1 |
| | pVSR326MOD | 10 | 8 | 0.8 |
| III | pVTPA-aadA | 10 | 16 | 1.6 |
| | pVSR326MOD | 10 | 10 | 1.0 |
| Total | pVTPA-aadA | 30 | 46 | 1.5 ± 0.4 |
| | pVSR326MOD | 30 | 31 | 1.0 ± 0.3 |
| Negative controls | — | 20 | 4 | 0.2 |
| (bombarded | | | | |
| without plasmid) | | | | |

The obtained experimental results confirm our hypothesis that, without carrying terminator sequences of plastid origin, the vector aim of this invention provides a high frequency of transplastomic plants production. Additionally, it was demonstrated the possibility that offers this vector for the expression of a foreign gene (in this case the aadA gene) under the transcriptional control of two promoters in tandem, PrbcL and Prrn.

### Example No. 4

### Expression of a Heterologous Bi-Cistronic Unit in Tomato Transplastomic Plants (pVTPA-f-GUS-aadA)

The vector aim of this invention offers the possibility of expressing several genes in form of policistronic unit. With the purpose of illustrating this possibility, in vector pVTPA-f-GUS (see Example No. 2) was inserted the aadA gene downstream of the gus gene to form a tri-cistronic unit (considering that the hgh marker gene will be also expressed as a part of the mRNA produced from the rbcL promoter), that after the elimination of the hgh selection marker by homologous recombination, will express only the genes that encode for the β-glucuronidase and the aminoglycoside 3′-adenylyltransferase.

The aadA gene was amplified by PCR as described in Example No. 3, and cloned in the ApaI site (Klenow blunted) of the plasmid pVTPA-f-GUS, giving rise to the vector pVTPA-f-GUS-aadA (SEQ ID NO: 23). This new vector was used for obtaining tomato transplastomic plants according to the methods described below:

Sterilized tomato seeds (var. Campbell-28) were germinated for 12 days on MSO medium (MS salts, Gamborg B5 vitamins, sucrose 30 g/L, phytoagar 8 g/L, pH 5.8) half diluted, and the cotyledonal leaves were collected in liquid MSO medium and cultivated with the abaxial side down in a petri dish with solid MSO medium supplemented with 1 mg/L of zeatin and 0.4 M of manitol for the osmotic pre-treatment of 4 hours. Transformation was performed by biolistic method as described in Example No. 2 for tobacco. The bombarded cotyledonal leaves were maintained in the same medium for 14-16 hours and later were placed with the abaxial side up on MSO medium supplemented with 1 mg/L of zeatin for two days. Subsequently, the bombarded cotyledonal leaves were transferred with the abaxial side up to MSO medium supplemented with zeatin 1 mg/L and Hygromycin-B 5 mg/L (Duchefa, Netherlands). Resistant green shoots and calli appear in 3-6 weeks of culture and are transferred to the same selective medium for a second selection cycle with a Hygromycin-B concentration of 10 mg/L. After 3-4 weeks, the resistant green shoots and calli were transferred to MSO medium with zeatin 0.1 mg/L and Hygromycin-B 15 mg/L, and 3 weeks later were rooted on MSO medium supplemented with Hygromycin-B 15 mg/L. All the culture stages were performed with a regime of 16 hours of light and 8 hours of darkness. The resistance to 500 mg/L of Spectinomycin (Spc^{r}) as well as GUS gene expression (by histochemical assay), were evaluated in the obtained clones. The results of three transformation experiments are shown in the following table:

TABLE 4
Obtainment of tomato transplastomic plants with the construction
pVTPA-f-GUS-aadA.

| | No. of | No. of | | |
|---|---|---|---|---|
| | bombarded | Hygromycin | | |
| | cotyledonal | resistant | Spc^{r} | GUS⁺ |
| Experiment. | leaves. | clones. | clones. | clones. |
| I | 75 | 6 | 6 | 6 |
| II | 100 | 9 | 9 | 9 |
| III | 100 | 7 | 7 | 7 |
| Total | 275 | 22 | 22 | 22 |

When plants were cultured under greenhouse conditions, it was not appreciated phenotype differences among transplastomic plants and non-transformed plants of tomato, so it was confirmed the functioning of hybrids atpB and rbcL proteins, and the feasible of genetically manipulating these proteins for improving the metabolic activity of plastids. This example illustrates not only the capacity of the vector aim of this invention for expressing various genes as part of the same mRNA, but besides, confirms the universality of these vectors as vehicles for obtaining transplastomic Angiosperm plants different from those plants used as donors of the border sequences for integration of heterologous DNA into plastid genomes.

### Example No. 5

### Expression of Two Independent Transcriptional Units in Tobacco Transplastomic Plants (pVTPA-HB-aadA). Obtainment of Transplastomic Plants that Express a Multimeric Vaccine Antigen

The gene that encodes for the hepatitis B surface antigen (HBsAg) (Valenzuela P; Gray P; Quiroga M; Zaldivar J; Goodman H. M; Rutter W. J. Nature 1979, 280:815-819) was obtained from plasmid pSAO503 (CIGB, Cuba) by digestion with EcoRI(Klenow blunted)/NcoI and inserted into NcoI/SmaI-digested pVIEP-2 vector (see Example 2) for fusing a ribosome binding site upstream of the HBsAg translation initiation codon, obtaining this way the plasmid pVIEP-2-HB. The DNA fragment of 820 bp comprising the gene that encodes for hepatitis B surface antigen was obtained from plasmid pVIEP-2-HB by digestion with XhoI and cloned upstream of the aadA gene in SalI-digested pVTPA-aadA vector (see Example No. 3), to obtain the vector pVTPA-HB-aadA (SEQ ID NO: 24), that carries the HbsAg gene in correct orientation under the transcriptional control of the rice rbcL promoter and also expresses the marker genes aadA and hgh under the Prrn promoter.

The vector pVTPA-HB-aadA was used for obtaining tobacco transplastomic plants that express the hepatitis B surface antigen, according to the transformation protocol described in Example No. 2. The selection of transplastomic plants was done initially with Hygromycin-B 10 mg/L, and finally was checked the resistance to Spectinomycin 500 mg/L, and the expression of the HbsAg by an ELISA specific assay (CIGB, Cuba) and Western blot (FIG. 6). The obtained results are shown in Table 5:

TABLE 5
Obtainment of tobacco transplastomic plants with the construction
pVTPA-HB-aadA.

| | | No. of | | No. of clones |
|---|---|---|---|---|
| | No. of | Hygromycin | | expressing the |
| | bombarded | resistant | | HBsAg |
| Experiment. | leaves. | clones. | Spc^{r} clones. | (ELISA). |
| I | 10 | 13 | 13 | 9 |
| II | 10 | 11 | 11 | 7 |
| Total | 20 | 24 | 24 | 16 |

These results confirm the usefulness of the vector aim of this invention for expressing two or more genes as independent transcriptional units in the genome of transplastomic plants. Additionally, it has been confirmed the utility of this vector to express in plastids genes that encode for useful proteins that form multimeric complexes. In particular, we achieved the expression of the surface multimeric antigen of hepatitis B virus. It is obvious that what is illustrated here is applicable to other vaccine antigens, and that the same, once expressed in plant cells, can be formulated as preparations able to induce immunity against these infectious agents when provided to human being or animals. Others multimeric proteins that could be expressed by this vector are the immunoglobulins.

### Example No. 6

### Expression of an Heterologous Gene in Plastids Under the Control of Two Promoters and a Mini-Cistron (pVTPA-GUS1-aadA and pVTPA-GUS3-aadA)

The intermediary vector for cloning and expression of genes in the vectors aim of the present invention, pVIEP (see Example No. 2), allows cloning a gene under the control of PpsbA* promoter and a mini-cistron designed for increasing the stability and promoting the translation of produced mRNA. This was used for cloning the gene uidA as described in Example No. 2 to obtain the plasmid pVIEP-GUS1.

For checking the functionality of the mini-cistron, it was made a genetic construction in which mini-cistron was eliminated from the plasmid pVIEP by digestion with SnaBI and NdeI, blunted with an S1 nuclease treatment and re-ligated to obtain the pVIEP-3 vector; where the uidA gene was cloned as described previously giving rise to plasmid pVIEP-3. Both expression cassettes containing the gus gene were obtained from intermediary vectors by HindIII digestion and cloned into pVTPA-aadA vector. The constructions finally obtained contain the gus gene under the control of rice rbcL and PpsbA* promoters, and they were named pVTPA-GUS1-aadA and pVTPA-GUS3-aadA respectively. Plastids of tobacco plants were transformed with these plasmids as described in Example No. 2.

Transplastomic plants were obtained with both constructions, and we made a comparison of GUS expression levels of 5 clones from each construction by the fluorimetric method using 4-MUG, according to the protocol described by Jefferson (Jefferson R. A. Plant Mol. Biol. Rep. 1987, 5: 387-405). The results are shown in Table 6.

TABLE 6
Expression of uidA gene in tobacco plastids under control
of two promoters and a mini-cistron.

| | | GUS activity (pM | |
|---|---|---|---|
| | | 4-MU/min × μg | |
| Construction. | Clones. | of total proteins). | Average ± SD |
| pVTPA-GUS1-aadA | GUS1-1.3.1 | 2490 | 2475 ± 903 |
| | GUS1-3.2.2 | 1736 | |
| | GUS1-9.1.2 | 4125 | |
| | GUS1-18.1.l | 957 | |
| | GUS1-23.2.l | 3068 | |
| pVTPA-GUS3-aadA | GUS3-2.1.3 | 517 | 978 ± 300 |
| | GUS3-7.1.1 | 1174 | |
| | GUS3-11.2.1 | 891 | |
| | GUS3-19.2.3 | 1532 | |
| | GUS3-21.1.1 | 775 | |

As the previous table shows, it was obtained high levels of GUS activity with both constructions that express the uidA gene under control of rice rbcL and PpsbA* promoters in tandem. Additionally, it was shown that the GUS activity average values in the transformed plants with the genetic construction that contain the mini-cistron are significantly higher, so it is demonstrated the functionality of this genetic element in plastids and its usefulness as a tool for increasing gene expression in this plant organelle.

### Example No. 7

### Obtainment of Rice Transplastomic Plants with the Construction pVTPA-f-GUS-aadA

Rice transplastomic plants were obtained by using the following protocol:

Rice embryogenic calli were produced from sterilized rice mature seeds cultured in N6-2 medium (Salts and vitamins of N6-2 medium (Chu C. C; and others. Scientia Sinic 1975, 18:659); myo-inositol 0.1 g/L; casein hydrolysate 1 g/L; 2,4D 2 mg/L; sucrose 30 g/L; phytagel 3 g/L, pH 5.7) for 21 days. These calli were sub cultivated on fresh N6-2 medium for 5 days, and later were placed on N6-2 medium supplemented with 3 mg/L of kinetin for 48-72 hours in the darkness. Before bombardment embryogenic calli are sub cultivated in the same medium supplemented with manitol 0.4 M for the osmotic pre-treatment. Approximately 30 calli were placed on a petri dish and gold particles (0.6 μm) coated with plasmid pVTPA-f-GUS-aadA were used for bombardment as described in Example No. 2. After bombardment calli remain in the same osmotic medium for 16 hours in the darkness and subsequently are sub cultivated in N6-2 medium for 2 days in the same conditions. Later, calli are transferred to N6-2 medium supplemented with 20 mg/L of Hygromycin B. After 10 days, antibiotic resistant calli are transferred to the same fresh medium for a second selection cycle for 10 days. Finally, resistant calli are transferred to a modified KIBAN regeneration medium (Salts and vitamins of MS medium; kinetin 3 mg/L; BAP 0.5 mg/L; NAA 1 mg/L; maltose 30 g/L; phytagel 4.5 g/L; pH 5.8) supplemented with 30 mg/L of Hygromycin B, and the obtained green shoots of approximately 2 cm after 3-4 weeks of culture in a photoperiod of 16 hours of light and 8 hours of darkness, are transferred to a micro propagation cycle in modified liquid MS medium (Salts and vitamins of MS medium; sucrose 30 g/L; BAP 5 mg/L; pH 5.7) with 20 mg/L of Hygromycin B. Shoots are placed in erlenmeyers with 50 mL of this liquid medium and incubated at 28° C. with agitation of 150 rpm and a photoperiod of 16 hours of light and 8 hours of darkness. Approximately seven new shoots are obtained per each incubated shoot after 7 days of incubation, which are rooted on solid MS medium with g/L of sucrose and 30 mg/L of Hygromycin B.

The obtained transplastomic clones were evaluated for Spectinomycin resistance (500 mg/L), as well as the expression of gus gene by histochemical assay. In addition, it was proved that the pre-treatment of the cells with cytokinins prior to bombardment significantly increased the frequency of transplastomic plants production. The results of these transformation experiments are shown in Table 7.

TABLE 7
Obtainment of rice transplastomic plants (var. IAC-28)
with the construction pVTPA-f-GUS-aadA, with and
without the pre-treatment with cytokinins (kinetin 3 mg/L).

| | | No. of | | |
|---|---|---|---|---|
| | No. of bombarded | Hygromycin | SPC^{r} | GUS⁺ |
| Experiment. | plates. | resistant clones. | clones. | clones. |
| I | 10 | 2 | 2 | 2 |
| II | 10 | 1 | 1 | 1 |
| III + Kinetina | 10 | 7 | 7 | 7 |
| IV + Kinetina | 10 | 8 | 8 | 8 |

The present example confirms the universality and functioning of the vectors aim of the present invention by transforming monocotyledonous species.

### Example No. 8

### Obtainment of Sugar Cane Transplastomic Plants with the Construction pVTPA-f-GUS-aadA

The obtainment of sugar cane transplastomic plants was performed as described below:

Young sugar cane leaves of the basal part of culms from 6 month old field plants (var. Ja60-5) were cut into 0.5×1 cm segments, sterilized and cultivated under light on P+5 solid medium (Salts and vitamins of MS; myo-Inositol 100 mg/L; casein hydrolyzate 500 mg/L; sucrose 20 g/L; 2,4D 5 mg/L; phytagel 4 g/L; pH 5.7) supplemented with 3 mg/L of kinetin for 48 hours. Later, the explants were placed for 6-8 hours under light on the same medium supplemented with manitol 0.4 M for an osmotic pre-treatment before bombardment. Gold particles (0.6 μm) were coated with the plasmid pVTPA-f-GUS-aadA, and the bombardment was performed as described in Example No. 2 by using the PDS-1000/He system (Bio-Rad). The bombarded explants were maintained on the same medium for 24 hours in the dark, and later were placed on P+5 medium for 2 days, following by a culture in P+5 medium supplemented with 15 mg/L of Hygromycin B in the dark. Hygromycin resistant calli appear after 4 weeks of culture and are transferred to light for regeneration on P— solid medium (P+5 medium without 2,4D) with Hygromycin B. Resistant regenerated shoots are isolated and rooted on P-medium with 20 mg/L of Hygromycin B.

The Spectinomycin (500 mg/L) resistance and the expression of gus gene (by histochemical assay) was evaluated in the sugarcane obtained clones. The results of two transformation experiments are shown in Table 8:

TABLE 8
Obtainment of sugar cane transplastomic plants
with the construction pVTPA-f-GUS-aadA.

| | No. of bombarded | No. of Hygromycin | Spc^{r} | GUS⁺ |
|---|---|---|---|---|
| Experiment. | explants. | resistant clones. | clones. | clones. |
| I | 20 | 11 | 11 | 11 |
| II | 20 | 7 | 7 | 7 |

The present example confirms again the universality of the vectors aim of this invention as vehicle for the obtainment of transplastomic Angiosperm plants different from those plants, which serve as donors for the sequences used as recombination borders for the integration of heterologous DNA into plastid genomes.

### Example No. 9

### Obtainment of Transplastomic Plants with Desirable Agronomic Characters

The obtainment of transplastomic plants with new incorporated agronomic characters is an important goal in the fight against pests, diseases, and weeds, the increase of yield, the decrease of costs and better qualities of agricultural products, without the risk of spreading the new genes to the environment.

The gene pat (bar) of Streptomyces hygroscopicus (Thompson C. J; Movva N. R; Tizard R; Crameri R; Davies J. E; and others. EMBO Journal 1987, 6:2519-2523) that encodes for the phosphinothricin (active ingredient of BASTA™ herbicide) acetyl transferase, was obtained from plasmid pBS-Bar (C.I.G.B) by digestion with XbaI(S1 nuclease blunted) and BamHI, and was cloned into vector pVIEP-2 (see Example No. 2) treated with NcoI (S1 nuclease blunted) and BamHI-digested, to produce the plasmid pVIEP2-Bar. The 670 bp DNA fragment comprising the pat gene with a RBS obtained from pVIEP2-Bar by XhoI digestion was cloned into pVTPA vector to produce the construction pVTPA-Bar (SEQ ID NO: 25). Tobacco transplastomic plants were obtained according to the protocol described in Example No. 2, identifying the transformants by resistance to phosphinothricin (Duchefa, Netherlands) at 5 mg/L in the culture medium. The obtained results are shown in Table 9.

TABLE 9
Obtainment of tobacco transplastomic plants resistant
to herbicide with the construction pVTPA-Bar.

| | No. of | |
|---|---|---|
| | bombarded | No. of phosphinothricin |
| Experiment. | leaves. | resistant clones (5 mg/L). |
| I | 10 | 13 |
| II | 10 | 14 |
| Control | 10 | 0 |
| (transformed | | |
| without plasmid) | | |

It is obvious that the cloning and expression in the vectors aim of this invention of other genes that confer resistance to herbicides, as for example, glyphosate, asulam, sulphonilurea, imidazolinone and bromoxinil, will produce transplastomic plants resistant to these chemicals. Equally, it is obvious that with the use of the vector aim of this invention is feasible to clone and express in transplastomic plants genes that confer resistance to pests and diseases, as for example the genes that encodes for the protein crystals of Bacillus thuringiensis; or for proteins with anti-fungal activity like chitinases and glucanases; or with anti-abiotic stress properties like choline oxidase. The modification or introduction of genes that increase the efficiency of photosynthesis, or the quality of plant products, are some of the plant functions that can be improved with the use of the universal vector for Angiosperm plastid transformation that this invention proposes.

### Example No. 10

### Expression of Proteins of Non-Agronomic Use in Plastids

In the Example No. 5 we demonstrated the utility of the vector aim of this invention for stable introduction and expression of genes that encodes for multimeric proteins in Angiosperm plant plastids. Others proteins of medical, veterinary, or industrial use are equally feasible of being produced in transplastomic plants by means of our transformation/expression vector system.

For expressing in chloroplasts the fibrinolitic agent streptokinase, very useful for the treatment of thrombosis and heart attacks, the 1254 bp DNA fragment that encodes for the mature protein SKC-2 of Streptococus was excised from the pEKG-3 vector (Estrada M. P; Hemandez L; Perez A; Rodriguez P; Serrano R; Rubiera R; Pedraza A; and others. Bio/Technology 1992, 10:1138-1142) by EcoRI(S1 nuclease blunted) and BamHI digestion, and inserted into the vector pVIEP (see Example No. 2) NcoI(S1 nuclease blunted)/BamHI-digested, to obtain the plasmid pVIEP-Strep. The cassette for the expression of streptokinase gene in plastids under the control of the PpsbA* promoter and the mini-cistron, was obtained by SalI/XhoI digestion from the plasmid pVIEP-Strep, and was cloned into the pVTPA vector (see Example No. 1) SalI/XhoI-digested to construct the plasmid pVTPA-Strep (SEQ ID NO: 26).

Plastids of tobacco plants were transformed with the vector pVTPA-Strep as described in Example No. 2. The results obtained are shown in Table 10 (the fibrinolitic activity of streptokinase produced in some clones was determined by the casein/plasminogen plate technique (Estrada M. P; Hemandez L; Perez A; Rodriguez P; Serrano R; Rubiera R; Pedraza A; y otros. Bio/Technology 1992, 10:1138-1142).

TABLE 10
Expresion of Streptokinase in tobacco plastids.

| | No. of | | Fibrinolitic |
|---|---|---|---|
| No. of | Hygromycin | | activity |
| bombarded | resistant | | (UI/mg of |
| leaves. | clones. | Clones. | proteins). |
| 10 | 14 | Strep2.3.1 | 650 |
| | | Strep4.5.2 | 400 |
| | | Strep5.2.2 | 1500 |
| | | Strep9.2.2 | 200 |
| | | Strep12.3.1 | 350 |
| | | Negative control | 0 |

These results confirm the utility of the vectors aim of this invention for expressing genes that encode for useful proteins in plastids, and in particular proteins of pharmaceutical or veterinary use like thrombolytics, interferons, cytokines, growth factors, hormones, antigens, cell receptors and others. It is obvious that what have been exemplified here can be also applied to the expression in plastids of others kind of enzymes and proteins of industrial use like lipases, proteases, etc by using the transformation/expression vectors described in the present document.

### Example No. 11

### Expression of Immunoglobulins in Plastids

The DNA fragments that encode for the heavy and light chains of the monoclonal antibody CB-Hep.1 specific for HbsAg were obtained by NcoI/XbaI digestion from plasmids pHESΩHBsAgCL and pHESΩHBsAgCH (Nadia Ramirez, “Transgenic tobacco (Nicotiana tabacum L.): a system for production of an antibody anti-HBsAg and its fragments”, PhD. Thesis, C.I.G.B., 2002) respectively, and inserted in the pVIEP vector NcoI/XbaI-digested for producing the plasmids pVIEP-Hc and pVIEP-Lc. Subsequently, the DNA fragment that encodes for the light chain was excised from pVIEP-Lc plasmid by SnaBI/BamHI digestion and inserted into pVIEP-Hc plasmid SmaI/BamHI-digested, giving rise to the bi-cistronic construction pVIEP-HcLc. Further, the expression cassette comprising the heavy and light chains, each one fused to a RBS, was cloned into pVTPA-f vector as a HindIII fragment; so, it was obtained the plasmid pVTPA-f-HcLc. This plasmid was used for the transformation of tobacco plastids as described in Example No. 2.

The obtained transplastomic plants were analyzed by Western blot for detecting the presence of both chains of the CB-Hep.1 monoclonal antibody (FIG. 7); and additionally it was proved the functioning of the produced recombinant immunoglobulins by using protein extracts from transplastomic plants to evidence the presence of the HbsAg in an ELISA that was revealed with an rabbit anti-mouse policlonal antibody conjugated to alkaline phosphatase.

This experiment demonstrated the possibility to produce completely functional antibodies in transplastomic plants by usisng our transformation/expression vectors, and at the same time we re-affirmed the possibility to express and correctly assemble multimeric proteins in plastids.

What is claimed is:

1. A DNA vector useful for stable transformation and expression of genes in plastids, where the gene to be expressed is inserted an the artificial intergenic region constituted by the combination of two 5′ untranslated regions of plastid genes whose transcription naturally occurs in opposite directions and that belong to plants of different Classes or Divisions.

2. A DNA vector as claimed in claim 1, useful for achieving a high frequency of stable transformation and expression of genes in plastids of Angiosperm plants, with the following features:
a) contains an artificial intergenic region where it is possible to insert DNA sequences that encode for a polypeptide, under appropriated signals for its transcription and translation in plastids using transcription terminator sequences of non-plastid origin,
b) the artificial intergenic region is flanked by two genes whose transcription naturally occurs in opposite directions in plastid genomes, and whose nucleotide sequences are identical to the encoding sequences of atpB and rbcL genes belonging to plastids of Angiosperm plants from different Classes (dicotyledonous and monocotyledonous),
c) the artificial intergenic region is composed by: 5′ regulatory sequences for transcription and translation of the atpB and rbcL genes; restriction sites for inserting genes of interest followed by a transcription terminator of non-plastid origin; and a second 5′ regulatory region for the transcription and translation of an rbcL gene belonging to Angiosperm plastids from plants of a Class different to the Class of the previous rbcL gene.

3. A DNA vector as claimed in claim 2, comprising a border DNA sequence homologous to the sequence of plastid rbcL gene from tobacco or rice.

4. A DNA vector as claimed in claim 3, wherein said DNA sequence homologous to the rbcL gene comprises at least a fragment of the nucleotide sequence from position −291 to position +1233, starting from the translation initiation codon, of the tobacco rbcL gene.

5. A DNA vector as claimed in claim 4, wherein said border sequence homologous to the rbcL gene comprises the sequence SEQ.ID.NO: 4.

6. A DNA vector as claimed in claim 2, comprising a border DNA sequence homologous to the sequence of plastid atpB gene from rice or tobacco.

7. A DNA vector as claimed in claim 6, wherein said DNA sequence homologous to the atpB gene comprises at least a fragment of the nucleotide sequence from position −654 to position +1211, starting from the translation initiation codon, of the rice atpB gene.

8. A DNA vector as claimed in claim 7, wherein said border sequence homologous to the atpB gene comprises the sequence SEQ.ID.NO: 8.

9. A DNA vector as claimed in claim 2, wherein said transcription terminator of non-plastid origin present in the artificial intergenic region, is the bi-directional terminator rrnBT1T2 from Escherichia coli or an homologous thereof.

10. A DNA vector as claimed in claim 2, having inserted in the artificial intergenic region a gene that allows the selection of transplastomic plants.

11. A DNA vector as claimed in claim 10, wherein said selection gene can be eliminated by homologous recombination between direct repeated DNA sequences that border the gene.

12. A DNA vector as claimed in claim 11, wherein said selection gene encodes for a protein that allows plant cells to survive in presence of an antimicrobial compound.

13. A DNA vector as claimed in claim 12, wherein said antimicrobial compound belongs to the group of aminoglycoside antibiotics.

14. A DNA vector as claimed in claim 13, wherein said antibiotic compound is the Hygromycin B.

15. A DNA vector as claimed in claim 13, wherein said antibiotic compound is the Spectinomycin.

16. A DNA vector as claimed in claim 12, wherein said antimicrobial compound belongs to the group of sulfonamides.

17. A DNA vector as claimed in claim 16, wherein said antimicrobial compound is the Sulfadiazine.

18. A DNA vector as claimed in claim 10, wherein said selection gene encodes for a protein that allows plant cells to survive in presence of an herbicide compound.

19. A DNA vector as claimed in claim 18, wherein said herbicide compound belongs to the group of glutamine synthetase inhibitors.

20. A DNA vector as claimed in claim 19, wherein said herbicide compound is the Glufosinate (phosphinothricin).

21. A DNA vector as claimed in claim 19, wherein said herbicide compound is the Bialaphos.

22. A DNA vector as claimed in claim 18, wherein said herbicide compound belongs to the group of 5-enolpiruvil-shikimate-3-phosphate synthase (EPSPS) inhibitors.

23. A DNA vector as claimed in claim 22, wherein said herbicide compound is the Glyphosate (N-phosphomethyl glycine).

24. A DNA vector as claimed in claim 18, wherein said herbicide compound belongs to the group of inhibitors of folate synthesis.

25. A DNA vector as claimed in claim 24, wherein said herbicide compound is the Asulam (methyl-(4-aminobencenosulphonyl)-carbamate).

26. A DNA vector as claimed in claim 18, wherein said herbicide compound belongs to the group of inhibitors of acetolactate synthase (ALS).

27. A DNA vector as claimed in claim 26, wherein said herbicide compound is a sulphonylurea.

28. A DNA vector as claimed in claim 26, wherein said herbicide compound is a imidazolinone.

29. A DNA vector as claimed in claim 18, wherein said herbicide compound belongs to the group of inhibitors of photosystem II.

30. A DNA vector as claimed in claim 29, wherein said herbicide compound is the Bromoxinyl (3,5-dibromo-4-hydroxibenzonitrile).

31. A DNA vector as claimed in claim 10, wherein said selection gene encodes for a protein that allows plant cells to survive in presence of a toxic compound.

32. A DNA vector as claimed in claim 31, wherein said toxic compound is the betaine aldehyde.

33. A DNA vector as claimed in claim 1, having the general structure described in FIG. 1-A or variants thereof.

34. A DNA vector as claimed in claims 1, having the general structure described in FIG. 1-B or variants thereof.

35. A DNA vector as claimed in claims 1, having the general structure described in FIG. 1-C or variants thereof.

36. A DNA vector as claimed in claim 1, having the general structure described in FIG. 1-D or variants thereof.

37. A DNA vector as claimed in claim 1, having the general structure described in FIG. 1-E or variants thereof.

38. A DNA vector as claimed in claim 1, having the general structure described in FIG. 1-F or variants thereof.

39. A DNA vector as claimed in claim 2, having in the second 5′ untranslated region of rbcL gene a sequence functionally homologous to the binding site of the Escherichia coli lacI repressor.

40. A DNA vector having upstream of the translation initiation codon of rbcL gene a DNA sequence that allows to repress the transcription of this gene in non induction conditions, in order to provide the maintenance and multiplication of the vector in any host.

41. A DNA vector as claimed in claim 40, having in the 5′ untranslated region of rbcL gene a sequence functionally homologous to the binding site of the Escherichia coli lacI repressor.

42. A DNA vector as claimed in claim 41, where the nucleotide sequence of said lacI binding site, corresponds at least in part to the sequence described in SEQ.ID.NO: 3.

43. A DNA vector as claimed in claim 1, having inserted in the artificial intergenic region one or more monocistronic or policistronic transcriptional units under one or more plastid functional promoters.

44. A DNA vector as claimed in claim 12, having inserted in the artificial intergenic region one or more monocistronic or policistronic transcriptional units under one or more plastid functional promoters.

45. A DNA vector as claimed in claim 18, having inserted in the artificial intergenic region one or more monocistronic or policistronic transcriptional units under one or more plastid functional promoters.

46. A DNA vector as claimed in claim 43, wherein at least one of the said plastid functional promoters is an rbcL promoter of the artificial intergenic region.

47. A DNA vector as claimed in claim 46, wherein the plastid functional promoters is the rice rbcL promoter comprised in sequence SEQ.ID.NO: 8.

48. A DNA vector as claimed in claim 43, wherein at least one of the said plastid functional promoters is the psbA* promoter comprised in the sequence SEQ.ID.NO: 16 or variants thereof.

49. A DNA vector as claimed in claim 48, having a mini-cistron functionally linked to said psbA* promoter.

50. A DNA vector as claimed in claim 49, wherein said mini-cistron have a nucleotide sequence homologous to sequence described in SEQ.ID.NO: 17 or variants thereof.

51. A DNA vector for stable introduction and expression of genes in plastids that uses a mini-cistron for increasing the expression of genes introduced into transplastomic plants.

52. A DNA vector as claimed in claim 43, wherein at least one gene of agricultural, veterinary, pharmaceutical, food or industrial interest is a component of the said transcriptional units.

53. A DNA vector as claimed in claim 52, wherein said gene of agricultural interest encodes for a protein with insecticide activity.

54. A DNA vector as claimed in claim 53, wherein said insecticide protein belongs to the family of Cry proteins from Bacillus thuringiensis.

55. A DNA vector as claimed in claim 53, wherein said insecticide protein is a protease inhibitor.

56. A DNA vector as claimed in claim 52, wherein said gene of agricultural interest encodes for a polypeptide with antimicrobial activity.

57. A DNA vector as claimed in claim 56, wherein said polypeptide belongs to one of the groups of the plant proteins related to pathogenicity (PR-proteins).

58. A DNA vector as claimed in claim 57, wherein said PR-protein is a glucanase.

59. A DNA vector as claimed in claim 57, wherein said PR-protein is a chitinase.

60. A DNA vector as claimed in claim 57, wherein said PR-protein is a thaumatin-like protein.

61. A DNA vector as claimed in claim 56, wherein said polypeptide is a ribosome inactivation protein (RIP).

62. A DNA vector as claimed in claim 52, wherein said gene of agricultural interest encodes for a protein that confers resistance to abiotic stress.

63. A DNA vector as claimed in claim 62, wherein said protein has choline oxidase activity.

64. A DNA vector as claimed in claim 52, wherein said gene of agricultural interest encodes for a protein that contributes to increase plant productivity.

65. A DNA vector as claimed in claim 64, wherein said protein is involved in the improvement of photosynthetic performance of the plant.

66. A DNA vector as claimed in claim 65, wherein said protein has fructose-1,6-bisphosphate phosphatase (FBPase) activity.

67. A DNA vector as claimed in claim 65, wherein said protein has protoporphyrinogen oxidase (PROTOX) activity.

68. A DNA vector as claimed in claim 65, wherein said protein has ribulose-1-5-bisphosphate carboxylase/oxygenase (RUBISCO) activity.

69. A DNA vector as claimed in claim 65, wherein said protein takes part directly or indirectly in the carbon fixation process of the plants.

70. A DNA vector as claimed in claim 52, wherein said gene of agricultural interest encodes for a protein that contributes to increase the post-harvest conservation of the plant products.

71. A DNA vector as claimed in claim 52, wherein said gene of food interest encodes for a protein that contributes to improve the nutritional quality of the plant products.

72. A DNA vector as claimed in claim 52, wherein said gene of veterinary or pharmaceutical interest encodes for a cytokine.

73. A DNA vector as claimed in claim 72, where said cytokine belongs to the family of interferons.

74. A DNA vector as claimed in claim 72, where said cytokine belongs to the family of interleukines.

75. A DNA vector as claimed in claim 52, wherein said gene of veterinary or pharmaceutical interest encodes for a polypeptide with regulatory activity on the immune response.

76. A DNA vector as claimed in claim 52, wherein said gene of veterinary or pharmaceutical interest encodes for a polypeptide with hormonal activity.

77. A DNA vector as claimed in claim 76, wherein said polypeptide with hormonal activity is the insulin.

78. A DNA vector as claimed in claim 76, wherein said polypeptide with hormonal activity is a growth hormone.

79. A DNA vector as claimed in claim 76, wherein said polypeptide with hormonal activity is a somatotropic hormone.

80. A DNA vector as claimed in claim 76, wherein said polypeptide with hormonal activity is a gonadotropic hormone.

81. A DNA vector as claimed in claim 52, wherein said gene of veterinary or pharmaceutical interest encodes for a cell proliferation factor.

82. A DNA vector as claimed in claim 81, wherein said cell proliferation factor is the epidermic growth factor (EGF).

83. A DNA vector as claimed in claim 52, wherein said gene of veterinary or pharmaceutical interest encodes for a polypeptide with hematopoietic activity.

84. A DNA vector as claimed in claim 52, wherein said gene of veterinary or pharmaceutical interest encodes for a cell receptor.

85. A DNA vector as claimed in claim 52, wherein said gene of veterinary or pharmaceutical interest encodes for a protease inhibitor.

86. A DNA vector as claimed in claim 52, wherein said gene of veterinary or pharmaceutical interest encodes for a polypeptide with trombolytic activity.

87. A DNA vector as claimed in claim 86, wherein said polypeptide is the streptokinase.

88. A DNA vector as claimed in claim 86, wherein said polypeptide is the tissular plasminogen activator (t-PA).

89. A DNA vector as claimed in claim 52, wherein said gene of veterinary or pharmaceutical interest encodes for a vaccine antigen.

90. A DNA vector as claimed in claim 89, wherein said vaccine antigen belongs to a virus.

91. A DNA vector as claimed in claim 90, wherein said vaccine antigen belongs to hepatitis virus.

92. A DNA vector as claimed in claim 91, wherein said vaccine antigen is the hepatitis B surface antigen.

93. A DNA vector as claimed in claim 91, wherein said vaccine antigen belongs to the hepatitis A virus.

94. A DNA vector as claimed in claim 91, wherein said vaccine antigen belongs to the hepatitis C virus.

95. A DNA vector as claimed in claim 90, wherein said vaccine antigen belongs to the foot and mouth disease virus (FMDV).

96. A DNA vector as claimed in claim 90, wherein said vaccine antigen belongs to the human immunodeficiency virus (HIV).

97. A DNA vector as claimed in claim 89, wherein said vaccine antigen belongs to a bacterium.

98. A DNA vector as claimed in claim 89, wherein said vaccine antigen belongs to a protozoan.

99. A DNA vector as claimed in claim 52, wherein said gene encodes for a fragment of the variable region of an immunoglobulin.

100. A DNA vector as claimed in claim 52, wherein said gene encodes for a multimeric protein.

101. A DNA vector as claimed in claim 100, wherein said multimeric protein is an immunoglobulin.

102. A DNA vector as claimed in claim 100, wherein said multimeric protein is a hormone.

103. A DNA vector as claimed in claim 100, wherein said multimeric protein is a vaccine antigen.

104. A DNA vector as claimed in claim 100, wherein said multimeric protein is an enzyme.

105. A DNA vector as claimed in claim 100, wherein said multimeric protein is a cell receptor.

106. A DNA vector as claimed in claim 52, wherein said gene of industrial interest encodes for a protein component of a biopolymer.

107. A DNA vector as claimed in claim 52, wherein said gene of industrial interest encodes for an enzyme.

108. A DNA vector as claimed in claim 107, wherein said enzyme is a protease.

109. A DNA vector as claimed in claim 107, wherein said enzyme is a lipase.

110. A DNA vector as claimed in claim 107, wherein said enzyme is an isomerase.

111. A DNA vector as claimed in claim 107, wherein said enzyme has glycosil-hydrolase activity.

112. A DNA vector as claimed in claim 111, wherein said enzyme with glycosil-hydrolase activity is a levansucrase.

113. A DNA vector as claimed in claim 111, wherein said enzyme with glycosil-hydrolase activity is an invertase.

114. A DNA vector as claimed in claim 111, wherein said enzyme with glycosil-hydrolase activity is a levanase.

115. A DNA vector as claimed in claim 111, wherein said enzyme with glycosil-hydrolase activity is a dextranase.

116. The culture of vegetable cells in presence of cytokinins, before introducing into them any of the DNA vectors claimed in claim 1, to increase the frequency of production of transplastomic plants.

117. The use of kinetin as claimed in claim 116.

118. The Angiosperm transplastomic plants stably transformed with the DNA vectors claimed in claim 1.

119. The progeny of transplastomic plants of claim 118.

120. The transplastomic plants of claim 18 that stably express at least one of the genes present in the DNA vector used for transformation.

121. The transplastomic plants of claim 118 with hybrid atpB and/or rbcL proteins.

122. The culture of transplastomic plants of claim 120.

123. The culture of cells of the transplastomic plants of claim 120.

124. The purification and use of the protein, or proteins, that produce the cells of the transplastomic plants of claim 120 as a result of the expression of said gene or genes.

125. The transplastomic plants of claim 118 that are Angiosperms.

126. The transplastomic plants of claim 125 that are dicotyledonous.

127. The transplastomic plants of claim 126 that are solanaceous.

128. The transplastomic plants of claim 127 that belong to one of the following species: tobacco, tomato or potato.

129. The transplastomic plants of claim 125 that are monocotyledonous.

130. The transplastomic plants of claim 129 that are gramineous.

131. The transplastomic plants of claim 130 that belong to one of the following species: rice, sugar cane, maize, wheat or barley.


SEQUENCE LISTING (26 sequences)

SEQ ID NO: 1 - DNA, 21 bp, Artificial Sequence
  Description of Artificial Sequence Oligonucleotide corresponding to the region -291 to -270 (from the start of translation) of the tobacco rbcL gene.
gggaagttcttattatttagg

SEQ ID NO: 2 - DNA, 20 bp, Artificial Sequence
  Description of Artificial Sequence Oligonucleotide corresponding to the region +1213 to +1233 (from the start of translation) of the tobacco rbcL gene.
ccaaggatgtcctaaagttc

SEQ ID NO: 3 - DNA, 133 bp, Artificial Sequence
  Description of Artificial Sequence Synthetic DNA fragment corresponding to the region -162 to -29 of the non-translating lider of the tobacco rbcL gene, with modifications to introduce an “ideal” lacO.
ccatggtctaataatcaaacattctgattagttgataattcaaattgtgagcgctcacaatttgaaagattcctgtgaaaagtttcattaacacggaattcgtgtcgagtagaccttgttgttgtgagaattc

SEQ ID NO: 4 - DNA, 1523 bp, Artificial Sequence
  Description of Artificial Sequence Nucleotide sequence of the rbcL-border.
gggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttgg

SEQ ID NO: 5 - DNA, 24 bp, Artificial Sequence
  Description of Artificial Sequence Oligonucleotide corresponding to the region -543 to -519 (from the start of translation) of the rice atpB gene.
gacttgagttgttgttattgtaag

SEQ ID NO: 6 - DNA, 23 bp, Artificial Sequence
  Description of Artificial Sequence Oligonucleotide corresponding to the region +1188 to +1211 (from the start of translation) of the rice atpB gene.
atgtcctgaagttctttgtaacg

SEQ ID NO: 7 - DNA, 132 bp, Artificial Sequence
  Description of Artificial Sequence Synthetic DNA fragment corresponding to the region -654 to -543 of the non-translating lider of the rice atpB gene, with added restriction sites.
aagcttggccaaaaaggccgtcgacaaaatggggggcatgcttaagttaatgaatatgtttcattcatataatatgtttcattcatatataatgggtacaccctgtgtacattctatgctataggaattcat

SEQ ID NO: 8 - DNA, 1887 bp, Artificial Sequence
  Description of Artificial Sequence Nucleotide sequence of the atpB-border.
aagcttggccaaaaaggccgtcgacaaaatggggggcatgcttaagttaatgaatatgtttcattcatataatatgtttcattcatatataatgggtacaccctgtgtacattctatgctataggaattcattcgacttgagttgttgttattgtaagttaacatgcttcgattattaaaccatggatttgattcaccaaatccatctttattgtatactctttaatagatatagcgcaaccccaaatcaacttctaatccttattaagttcttaatagacccccttttcttattttgagtggaaatacctaaatactacgaaaattctctgttgacagcaatctatgcttcacagtagtatatattttgtatatcgaagtcctagataagaaagtagagtaggcacaaatcgtttacaaaaggcaaaatgtatatgaaaaaaagattgattgaactttccgacggactcattccatgagtaaacgattgaatgggattcgtttgggcaacgaaatcaagtgctggtccccttttctctcttattgaattaactaattcatttccttttgacttttggatttttggatatttttttggtgttgatttggcattattcaacaagaaaaaaatcaaaatttcgataaattccttttttttgaaaattatgtgataattatgagaaccaatcctactacttctcgtcccggggtttctacaattgaagaaaaaagtacagggcgtatcgatcaaattattggacccgtgctggatgtcacttttcccccgggcaagttaccttatatttataatgctttggtagtcaagagtcgagacactgacggtaagcaaattaatgtaacttgtgaggtacaacaattattaggaaataatcgagttagagctgtagctatgagtgctacagatgggttgatgagaggaatggaagtgattgacacgggagctcctctcagtgttcctgtcggtggagctactcttggacgaattttcaacgttcttggggagcctgttgacaatttgggtcctgtagatactagtgcaacattccctattcatagatccgcgcccgcctttatcgagttagatacgaaattatccatctttgaaactggtattaaggtggtcgatcttttagctccttatcggcgtggaggaaaaatcggactatttgggggagctggagtaggtaaaacagtactcatcatggaattaatcaacaatattgctaaagctcacgggggcgtatccgtatttggcggagtaggggaacggactcgtgaaggaaatgatctttatatggaaatgaaggaatctggagtaattaatgaaaaaaatcttgaggaatcaaaggtagctctagtctatggccaaatgaatgaaccgccaggagctcgtatgagagttggtttgactgccctaactatggcagaatatttccgagatgttaataagcaagacgtgcttctattcatcgataatatctttcgttttgttcaagcaggatcggaggtatctgccttattagggagaatgccctctgcagtgggttatcaacctactcttagtacagaaatgggttctttgcaagaaagaattacttctactaaaaagggatctataacttcgatccaagcggtttatgtacctgcggacgatttgaccgaccctgctcctgctacaacatttgcacatttggatgctactaccgtactttccagaggattagcttccaaagggatttatcctgcagtagatcctttagattcaacctcaactatgttacaacctcggatcgttggcaacgaacattatgaaactgcacaaagagttaagcaaactttacaacgttacaaagaacttcaggacat

SEQ ID NO: 9 - DNA, 24 bp, Artificial Sequence
  Description of Artificial Sequence Oligonucleotide primer used to amplify the hgh gene by PCR, with a RBS.
gggaggaatgagatatgaaaaagc

SEQ ID NO: 10 - DNA, 24 bp, Artificial Sequence
  Description of Artificial Sequence Oligonucleotide primer used to amplify the hgh gene by PCR, with added KpnI site.
gtcggtacctactctatttctttg

SEQ ID NO: 11 - DNA, 41 bp, Artificial Sequence
  Description of Artificial Sequence Synthetic DNA fragment with useful restriction sites for the construction of the selection cassette with the hgh gene.
aagcttgattcgagtgaacgcgtatagggcccgggagatct

SEQ ID NO: 12 - DNA, 2223 bp, Artificial Sequence
  Description of Artificial Sequence Nucleotide sequence of the DNA fragment containing the selection cassette with repeated borders that was cloned in the pBluescript.
aagcttgattcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatccactagttctaga

SEQ ID NO: 13 - DNA, 5669 bp, Artificial Sequence
  Description of Artificial Sequence Nucleotide sequence of DNA fragment from the vector pVTPA-f between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggcctttttggccaagcttgattcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 14 - DNA, 176 bp, Artificial Sequence
  Description of Artificial Sequence Synthetic DNA fragment codifying for the promoter region of the plastid 16S ribosomal RNA (Prrn), with added restriction sites.
gaattcccccgggctgctcccccgccgtcgttcaatgagaatggataagaggctcgtgggattgacgtgagggggcagggatggctatatttctgggagcgaactccgggcgaatacgaagcgcttggatacagttgtagggagggatttcatcgtttaaactcgagtgaacgcgt

SEQ ID NO: 15 - DNA, 5834 bp, Artificial Sequence
  Description of Artificial Sequence Nucleotide sequence of DNA fragment from the vector pVTPA between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggcctttttggccaagcttgatatcgaattcccccgggctgctcccccgccgtcgttcaatgagaatggataagaggctcgtgggattgacgtgagggggcagggatggctatatttctgggagcgaactccgggcgaatacgaagcgcttggatacagttgtagggagggatttcatcgtttaaactcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 16 - DNA, 96 bp, Artificial Sequence
  Description of Artificial Sequence Nucleotide sequence of the chimeric PpsbA* promoter with added restriction sites and a RBS.
gaattcaccttggttgacacgagtatataagtcatgttatactgttgaataaaaagccttccattttgattaaataaaggaggattttcatatgat

SEQ ID NO: 17 - DNA, 106 bp, Artificial Sequence
  Description of Artificial Sequence Synthetic DNA fragment codifying for a mini-cistron, a RBS, and restriction sites.
catatgtatcgattacgtaaggaggaataaaccatggacgagctctagactgcagcatgcccgggatcctaggcctgatatcaagcttctcgagctgtcgacagct

SEQ ID NO: 18 - DNA, 365 bp, Artificial Sequence
  Description of Artificial Sequence Nucleotide sequence of DNA fragment from the vector pVIEP showing the expression cassette and surrounding restriction sites.
ggtaccgggccccccctcgaggtcgacggtatcgataagcttgatatcgaattcaccttggttgacacgagtatataagtcatgttatactgttgaataaaaagccttccattttgattaaataaaggaggattttcatatgtatcgattacgtaaggaggaataaaccatggacgagctctagactgcagcatgcccgggatccggctgctaacaaagcccgaaaggaagctgagttggctgctgccaccgctgagcaataactagcataaccccttggggcctctaaacgggtcttgaggggttttttgctgaaaggaggaactatatccggatcctgatatcaagcttctcgagctgtcgac

SEQ ID NO: 19 - DNA, 7510 bp, Artificial Sequence
  Description of Artificial Sequence Nucleotide sequence of DNA fragment from the vector pVTPA-f-GUS between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggcctttttggccaagcttgatatcggtaaggaggaataaaccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaattcgagctcggtaccccgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 20 - DNA, 33 bp, Artificial Sequence
  Description of Artificial Sequence Oligonucleotide primer used to amplify the aadA gene by PCR, with a RBS.
aaggagaataccatgggggaagcggtgatcgcc

SEQ ID NO: 21 - DNA, 29 bp, Artificial Sequence
  Description of Artificial Sequence Oligonucleotide primer used to amplify the aadA gene by PCR.
gaacgagctcttagacattatttgccgac

SEQ ID NO: 22 - DNA, 6659 bp, Artificial Sequence
  Description of Artificial Sequence Nucleotide sequence of DNA fragment from the vector pVTPA-aadA between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggcctttttggccaagcttgatatcgaattcccccgggctgctcccccgccgtcgttcaatgagaatggataagaggctcgtgggattgacgtgagggggcagggatggctatatttctgggagcgaactccgggcgaatacgaagcgcttggatacagttgtagggagggatttcatcgtttaaactcgaaaggagaataccatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaagagctcgttctcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 23 - DNA, 8327 bp, Artificial Sequence
  Description of Artificial Sequence Nucleotide sequence of DNA fragment from the vector pVTPA-f-GUS-aadA between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggcctttttggccaagcttgatatcggtaaggaggaataaaccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaattcgagctcggtaccccgcgtatagaaggagaataccatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaagagctcgttccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 24 - DNA, 7549 bp, Artificial Sequence
  Description of Artificial Sequence Nucleotide sequence of DNA fragment from the vector pVTPA-HB-aadA between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgaggtcgacggtatcgataagcttgatatcggtaaggaggaataaaccatggagaacatcacatcaggattcctaggacccctgctcgtgttacaggcggggtttttcttgttgacaagaatcctcacaataccgcagagtctagactcgtggtggacttctctcaattttctagggggatctcccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaagaattgggatccggctgctaacaaagcccgaaaggaagctgagttggctgctgccaccgctgagcaataactagcataaccccttggggcctctaaacgggtcttgaggggttttttgctgaaaggaggaactatatccggatcctgatatcaagcttctcgacggcctttttggccaagcttgatatcgaattcccccgggctgctcccccgccgtcgttcaatgagaatggataagaggctcgtgggattgacgtgagggggcagggatggctatatttctgggagcgaactccgggcgaatacgaagcgcttggatacagttgtagggagggatttcatcgtttaaactcgaaaggagaataccatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaagagctcgttctcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 25 - DNA, 6465 bp, Artificial Sequence
  Description of Artificial Sequence Nucleotide sequence of DNA fragment from the vector pVTPA-Bar between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggcctttttggccaagcttgatatcgaattcccccgggctgctcccccgccgtcgttcaatgagaatggataagaggctcgtgggattgacgtgagggggcagggatggctatatttctgggagcgaactccgggcgaatacgaagcgcttggatacagttgtagggagggatttcatcgtttaaactcgaggtcgacggtatcgataagcttgatatcggtaaggaggaataaacaatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatgacccgggggatcctgatatcaagcttctcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga

SEQ ID NO: 26 - DNA, 7057 bp, Artificial Sequence
  Description of Artificial Sequence Nucleotide sequence of DNA fragment from the vector pVTPA-Estrep between the rice atpB and tobacco rbcL borders.
gtcgaggtcatgtcctgaagttctttgtaacgttgtaaagtttgcttaactctttgtgcagtttcataatgttcgttgccaacgatccgaggttgtaacatagttgaggttgaatctaaaggatctactgcaggataaatccctttggaagctaatcctctggaaagtacggtagtagcatccaaatgtgcaaatgttgtagcaggagcagggtcggtcaaatcgtccgcaggtacataaaccgcttggatcgaagttatagatccctttttagtagaagtaattctttcttgcaaagaacccatttctgtactaagagtaggttgataacccactgcagagggcattctccctaataaggcagatacctccgatcctgcttgaacaaaacgaaagatattatcgatgaatagaagcacgtcttgcttattaacatctcggaaatattctgccatagttagggcagtcaaaccaactctcatacgagctcctggcggttcattcatttggccatagactagagctacctttgattcctcaagatttttttcattaattactccagattccttcatttccatataaagatcatttccttcacgagtccgttcccctactccgccaaatacggatacgcccccgtgagctttagcaatattgttgattaattccatgatgagtactgttttacctactccagctcccccaaatagtccgatttttcctccacgccgataaggagctaaaagatcgaccaccttaataccagtttcaaagatggataatttcgtatctaactcgataaaggcgggcgcggatctatgaatagggaatgttgcactagtatctacaggacccaaattgtcaacaggctccccaagaacgttgaaaattcgtccaagagtagctccaccgacaggaacactgagaggagctcccgtgtcaatcacttccattcctctcatcaacccatctgtagcactcatagctacagctctaactcgattatttcctaataattgttgtacctcacaagttacattaatttgcttaccgtcagtgtctcgactcttgactaccaaagcattataaatataaggtaacttgcccgggggaaaagtgacatccagcacgggtccaataatttgatcgatacgccctgtacttttttcttcaattgtagaaaccccgggacgagaagtagtaggattggttctcataattatcacataattttcaaaaaaaaggaatttatcgaaattttgatttttttcttgttgaataatgccaaatcaacaccaaaaaaatatccaaaaatccaaaagtcaaaaggaaatgaattagttaattcaataagagagaaaaggggaccagcacttgatttcgttgcccaaacgaatcccattcaatcgtttactcatggaatgagtccgtcggaaagttcaatcaatctttttttcatatacattttgccttttgtaaacgatttgtgcctactctactttcttatctaggacttcgatatacaaaatatatactactgtgaagcatagattgctgtcaacagagaattttcgtagtatttaggtatttccactcaaaataagaaaagggggtctattaagaacttaataaggattagaagttgatttggggttgcgctatatctattaaagagtatacaataaagatggatttggtgaatcaaatccatggtttaataatcgaagcatgttaacttacaataacaacaactcaagtcgaatgaattcctatagcatagaatgtacacagggtgtacccattatatatgaatgaaacatattatatgaatgaaacatattcattaacttaagcatgccccccattttgtcgacggtatcgataagcttgatatcgaattcaccttggttgacacgagtatataagtcatgttatactgttgaataaaaagccttccattttgattaaataaaggaggattttcatatgtatcgattacgtaaggaggaataaaccatgattgctggacctgagtggctgctagaccgtccatctgtcaacaacagccaattagttgttagcgttgctggtactgttgaggggacgaatcaagacattagtcttaaattttttgaaattgacctaacatcacgacctgctcatggaggaaagacagagcaaggcttaagtccaaaatcaaaaccatttgctactgatagtggcgcgatgccacataaacttgaaaaagctgacttactaaaggctattcaagaacaattgatcgctaacgtccacagtaacgacgactactttgaggtcattgattttgcaagcgatgcaaccattactgatcgaaacggcaaggtctactttgctgacaaagatggttcggtaaccttgccgacccaacctgtccaagaatttttgctaagcggacatgtgcgcgttagaccatataaagaaaaaccaatacaaaatcaagcgaaatctgttgatgtggaatatactgtacagtttactcccttaaaccctgatgacgatttcagaccaggtctcaaagatactaagctattgaaaacactagctatcggtgacaccatcacatctcaagaattactagctcaagcacaaagcattttaaacaaaacccacccaggctatacgatttatgaacgtgactcctcaatcgtcactcatgacaatgacattttccgtacgattttaccaatggatcaagagtttacttaccatgtcaaaaatcgggaacaagcttatgagatcaataaaaaatctggtctgaatgaagaaataaacaacactgacctgatctctgagaaatattacgtccttaaaaaaggggaaaagccgtatgatccctttgatcgcagtcacttgaaactgttcaccatcaaatacgttgatgtcaacaccaacgaattgctaaaaagcgagcagctcttaacagctagcgaacgtaacttagacttcagagatttatacgatcctcgtgataaggctaaactactctacaacaatctcgatgcttttggtattatggactataccttaactggaaaagtagaggataatcacgatgacaccaaccgtatcataaccgtttatatgggcaagcgacccgaaggagagaatgctagctatcatttagcctatgataaagatcgttataccgaagaagaacgagaagtttacagctacctgcgttatacagggacacctatacctgataaccctaacgacaaataaggatcctgatatcaagcttctcgagtgaacgcgtatagggcccaattcgagctcggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtcaagtgcggccttgttctgagtggtctcaattacggtagcagtacctaagccctcgggggatctggggaggaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtaggtaccagcaccaccagcggtgaggtgcggaacttctacaacctcaaagcccataacgttgcggatagaacccttctcagggtcaatcagagcagcgtagtttgctgcgttcggcatcagtgctgccagaatcgcagagtagctatctgggtcacagtagaacacacggtcagcagccggaacatagttcttggtcagagccgcacgagccttagtcagagccgcaataatctccttacccagcgcaacttggtcggtaagtgcggccttgttctgagtggtctcaattacggtagcagtacctaagccctcgggggatcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctgggggatcagcttgatggccgccagtgtgatggatgggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataattcaaattgtgagcgctcacaatttgaaagattcctgtgaaaagtttcattaacacggaattcgtgtcgagtagaccttgttgttgtgagaattcttaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggatctgcagctagttctaga